(19) United States
(12) Patent Application Publication
(10) Pub. No.: US 2003/0185753 A1
(43) Pub. Date: Oct. 2, 2003

(54) ELISA kit for the determination of CYP 2C9 metabolic phenotypes and uses therefor

(75) Inventor: Brian Leyland-Jones, Miami, FL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Xanthus Life Sciences, Inc., Cambridge, MA

(21) Appl. No.: 10/325,697

(22) Filed: Dec. 19, 2002

Related U.S. Application Data
(60) Provisional application No. 60/340,855, filed on Dec. 19, 2001.

(51) Int. Cl. A61K 49/00; G01N 33/574

(52) U.S. Cl. 424/9.1; 435/7.23

(57) ABSTRACT

The invention relates to an enzyme linked immunosorbent assay (ELISA) method and kit for the rapid determination of metabolic phenotypes for Cytochrome P450 2C9 (CYP 2C9). The kit uses may include but are not limited to, use on a routine basis in a clinical laboratory to determine a Cytochrome P450 2C9 (CYP 2C9) phenotype of an individual; to allow a physician to individualize an individual's treatment with respect to the numerous drugs metabolized by CYP 2C9 based on a phenotypic determination; to predict an individual's susceptibility to carcinogen induced diseases including many cancers, and to screen individuals for a preferred metabolic phenotype in order to determine those individuals with a responsive phenotype for participation in clinical testing.

### RELATED APPLICATION

This application is a new application which claims the benefit of U.S. Provisional Application No. 60/340,855, filed on Dec. 19, 2001. The entire teachings of the above applications are incorporated herein by reference.

### BACKGROUND OF THE INVENTION

The invention relates to an enzyme linked immunosorbent assay (ELISA) method and kit for the rapid determination of metabolic phenotypes for Cytochrome P450 2C9 (CYP 2C9). The kit uses may include but are not limited to, use on a routine basis in a clinical laboratory to determine a CYP 2C9-specific phenotype of an individual; to allow a physician to individualize an individual's treatment with respect to the numerous drugs metabolized by CYP 2C9 based on a phenotypic characterization of the individual; to predict an individual's susceptibility to carcinogen induced diseases including many cancers, and to screen individuals for a preferred metabolic phenotype in order to determine those individuals with a responsive phenotype for participation in clinical testing and/or for treatment with a particular drug or class of drug compounds.

For the majority of drugs (or xenobiotics) administered to humans, their fate is to be metabolized in the liver, into a form less toxic and lipophilic with their subsequent excretion in the urine. Their metabolism is involves two systems which act consecutively: the cytochrome P450 system which includes at least 20 enzymes catalyzing oxidation reactions and localized in the microsomal fraction, and the conjugation system which involves at least 5 enzymes. An enzyme of one system can act on several drugs and drug metabolites. The rate of metabolism of a drug differs between individuals and between ethnic groups, owing to the existence of enzymatic polymorphism within each system. As a result, a variety of phenotypes can be distinguished, including poor metabolizers (PM), extensive metabolizers (EM), and ultra-extensive metabolizers (UEM).

As described in U.S. Pat. No. 5,830,672, Applicants have previously been successful in establishing an ELISA based system and method for the rapid determination of N-acetyltransferase (NAT2) phenotypes. However, to date a convenient and effective system for determining CYP 2C9 phenotypes has not been provided.

In previous studies, CYP 2C9 phenotypes have been generally determined by determining the ratio of the probe substrate (s)-ibuprofen and its metabolite 2-carboxyibuprofen in an individual. In these studies, the individuals ingest a dose of (s)-ibuprofen, and the is urinary concentrations of the two compounds are determined by liquid chromatography/tandem mass spectrometry (LC/MS/MS) or high-pressure liquid chromatography (HPLC). Existing CYP2C9 determination methods are time-consuming, onerous, and employ systems and equipment which are not readily available in a clinical laboratory.

It would be highly desirable to be provided with a convenient and effective method for characterizing an individual's CYP 2C9 phenotype using a non-toxic substrate so as to predict his/her response and side effects profile to a wide range of potentially toxic drugs.

It would be highly desirable to be provided with an enzyme linked immunosorbent assay (ELISA) kit for CYP 2C9 phenotyping, which could be accomplished on a routine basis by any technician with a minimum of training and does not involve complex equipment.

It would also be highly desirable to be provided with an enzyme linked immunosorbent assay (ELISA) kit, which would enable a physician to individualize therapy and/or treatment. Such therapies may include treatment with drugs such as phenytoin, tolbutamide, and nonsteroidal anti-inflammatory drugs (NSAIDS) based on an individual's CYP 2C9-specific phenotype.

### SUMMARY OF THE INVENTION

One aim of the present invention is to provide an enzyme linked immunosorbent assay (ELISA) kit for the rapid determination of metabolic enzyme phenotype, which can be used on a routine basis in a clinical laboratory.

Another aim of the present invention is to provide an ELISA kit which allows a physician to:

a) determine the CYP 2C9 metabolic phenotype of an individual;

b) individualize therapies or treatments with drugs known to be dependent on CYP 2C9 metabolism, according to an individual's metabolic phenotype;

c) predict an individual's susceptibility to carcinogen induced diseases such as various cancers;

d) screen individuals for a preferred CYP 2C9 metabolic phenotype in order to determine those individuals with a responsive phenotype for participation in clinical testing.

Another aim of the present invention is to provide a method for determining an individual's metabolic enzyme phenotype in order to predict his/her responsiveness to a drug treatment regime.

The ELISA phenotyping kit according to an embodiment of the present invention employs at least one non-toxic substrate (or probe substrate) known to be metabolized by the CYP 2C9 pathway for the determination of the CYP 2C9 phenotypes.

According to one aspect of this invention there is provided a method of characterizing a CYP 2C9-specific phenotype, said method comprising (a) administering to an individual a substrate known to be metabolized by a CYP 2C9 metabolic pathway; (b) detecting metabolites of said metabolic pathway in a biological sample obtained from the individual at a predetermined time after the administering of said substrate; and (c) characterizing a phenotypic determinant based on said metabolites which is indicative of said CYP 2C9 phenotype.

According to another aspect of this invention there is provided a competitive enzyme linked immunosorbent assay (ELISA) method for determining a CYP 2C9 phenotype, which comprises using at least two antibodies specific to (s)-ibuprofen and 2-carboxyibuprofen respectively, to determine the amount of each of (s)-ibuprofen and 2-carboxyibuprofen respectively, in a biological sample obtained from an individual treated with (s)-ibuprofen; wherein a molar ratio based on amounts of the (s)-ibuprofen to 2-carboxyibuprofen is indicative of a CYP 2C9 phenotype of said individual.

According to another aspect of this invention there is provided a competitive enzyme linked immunosorbent assay (ELISA) method for determining a CYP 2C9 phenotype, which comprises using at least two antibodies specific to losartan and E-3174 respectively, to determine the amount of each of losartan and E-3174 respectively, in a biological sample obtained from an individual treated with losartan; wherein a molar ratio based on amounts of the losartan to E-3174 is indicative of a CYP 2C9 phenotype of said individual.

According to yet another aspect of this invention there is provided a competitive enzyme linked immunosorbent assay (ELISA) kit for determining a CYP 2C9 phenotype, which comprises at least two antibodies, one specific to (s)-ibuprofen and another specific to 2-carboxyibuprofen, for detecting their molar ratio in a biological sample of an individual after consuming a dose of (s)-ibuprofen wherein said molar ratio is indicative of the CYP2C9 phenotype of said individual.

According to yet another aspect of this invention there is provided a competitive enzyme linked immunosorbent assay (ELISA) kit for determining a CYP 2C9 phenotype, which comprises at least two antibodies, one specific to losartan and another specific to E-3174, for detecting their molar ratio in a biological sample of an individual after consuming a dose of losartan wherein said molar ratio is indicative of the CYP2C9 phenotype of said individual.

According to still a further aspect of this invention, the probe substrate to be used is a dose of (s)-ibuprofen. An individual to be phenotyped consumes the probe substrate, and the individual's urine is collected 4 hours after consumption. Urine samples are subsequently analyzed via the ELISA technology of the present invention. In particular, the urine samples are analysed for respective amounts of (s)-ibuprofen and 2-carboxyibuprofen and the ratio thereof is calculated. Based on this ratio, an individual's CYP 2C9 metabolic phenotype can be characterized.

According to still a further aspect of this invention, the probe substrate to be used is a dose of losartan. An individual to be phenotyped consumes the probe substrate, and the individual's urine is collected 4 hours after consumption. Urine samples are subsequently analyzed via the ELISA technology of the present invention. In particular, the urine samples are analysed for respective amounts of losartan and E-3174 and the ratio thereof is calculated. Based on this ratio, an individual's CYP 2C9 metabolic phenotype can be characterized.

According to yet a further aspect of the present invention there is provided derivatives of (s)-ibuprofen and 2-carboxyibuprofen and uses thereof.

According to yet a further aspect of the present invention there is provided derivatives of losartan and E-3174 and uses thereof.

The term “phenotypic determinant” is intended to mean a qualitative or quantitative indicator of an enzyme-specific capacity of an individual.

The term “individualization” as it appears herein with respect to therapy is intended to mean a therapy having specificity to at least an individual's phenotype as calculated according to a predetermined formula on an individual basis.

The term “biological sample” is intended to mean a sample obtained from a biological entity and includes, but is not to be limited to, any one of the following: tissue, cerebrospinal fluid, plasma, serum, saliva, blood, nasal mucosa, urine, synovial fluid, microcapillary microdialysis and breath.

### BRIEF DESCRIPTION OF THE DRAWINGS

FIG. 1 illustrates structures of (s)-ibuprofen and 2-carboxyibuprofen.

FIG. 2 illustrates structures of losartan and E-3174.

FIG. 3 illustrates (s)-ibuprofen derivatives for CYP 2C9 phenotyping by ELISA.

FIG. 4 illustrates 2-carboxyibuprofen derivatives for CYP 2C9 phenotyping by ELISA.

FIG. 5 illustrates losartan derivatives for CYP 2C9 phenotyping by ELISA.

FIG. 6 illustrates E-3174 derivatives for CYP 2C9 phenotyping by ELISA.

FIG. 7 illustrates a pattern of samples to be added to a 96-well microtest plate.

### DETAILED DESCRIPTION OF THE INVENTION

CYP 2C9

The CYP2C9 family of metabolic enzymes accounts for approximately 8% of the metabolic enzymes in the liver. CYP 2C9 has been postulated as participating in approximately 15% of drug metabolism. Accordingly, the ability to determine an individual's capacity for CYP 2C9-specific metabolism prior to treatment with a drug known to be metabolized, at least in part by the CYP 2C9 pathway would be advantageous. Furthermore, the ability to determine a CYP 2C9-specific phenotype according to the present invention will allow for the individualization of therapy with CYP 2C9-specific treatments.

Polymorphism

Individuals are genetically polymorphic with respect to CYP 2C9 metabolism. Two metabolic phenotypes can be distinguished: extensive and poor metabolizers. Three genetic polymorphisms have been definitively identified, one wild type (CYP2C9*1) and two mutants (CYP2C9*2 and CYP2C9*3). The CYP2C9*2 allele was found to result in 5- to 10-fold increase in expression of mRNA and have a 3-fold higher enzyme activity for metabolism of phenytoin and tolbutamide. Conversely, this genotype appears to have a lower level of activity for the metabolism of S-warfarin. The CYP2C9*3 allele appears to demonstrate decreased metabolic activity against all three of these substrates.

CYP 2C9 metabolizes a variety of compounds including S-warfarin, phenytoin, tolbutamide, tienilic acid, and a number of nonsteroidal anti-inflammatory drugs such as diclofenac, piroxicam, tenoxicam, ibuprofen, and acetylsalicylic acid. The following table (Table 1) provides a much more detailed listing of CYP 2C9 substrates.

TABLE 1
CYP 2C9 Substrates

| Category | Subcategory | Chemical/Drug |
|---|---|---|
| Analgesic, antipyretic, | NSAID, Propionic acid deriv., | Aceclofenac |
| anti-inflammatory | Cyclooxygenase inhibitor | |
| Analgesic, anti pyretic, | Analgesic, Antipyretic, p- | Acetaminophen, Paracetamol |
| anti-inflammatory | Aminophenol | |
| Analgesic, antipyretic, | Analgesic, Opioid, | Acetylmethadol, L-alpha |
| anti-inflammatory | Diphenylheptane | |
| Analgesic, antipyretic, | Analgesic, Opioid, | Acetylmethadol, nor-L-alpha |
| anti-inflammatory | Diphenylheptane | |
| Analgesic, antipyretic, | NSAID, Antiplatelet, Salicylate, | Acetylsalicylic acid, Aspirin |
| anti-inflammatory | Cyclooxygenase inhibitor | |
| Analgesic, antipyretic, | NSAID, Pyrazolone, | Aminopyrine, Amidopyrine |
| anti-inflammatory | Cyclooxygenase inhibitor (less | Aminophenazone |
| | potent) | |
| Analgesic, antipyretic, | NSAID, Pyrazolone, | Antipyrine, Phenazone |
| anti-inflammatory | Prostaglandin synthesis inhibitor | |
| Analgesc antipyretic | NSAID Cclooxygenase-II | Celecoxib |
| anti-inflammatory | inhibitor | |
| Analgesic, antipyretic, | NSAID, Phenylacetic acid deriv., | Diclofenac (used as |
| anti-inflammatory | Cyclooxygenase-II inhibitor | test/marker substrate) |
| Analgesic, antipyretic, | NSAID, Phenylacetic acid deriv., | Diclofenac, 5-hydroxy |
| anti-inflammatory | Cyclooxygenase-II inhibitor | |
| Analgesic, antipyretic, | NSAID, Phenylacetic acid deriv., | Diclofenac, CH(2)OH |
| anti-inflammatory | Cyclooxygenase-lI inhibitor | derivative |
| Analgesic, antipyretic, | NSAID, Cyclooxygenase-II | Etoricoxib |
| anti-inflammatory | inhibitor | |
| Analgesic, antipyretic, | NSAID, Propionic acid deriv, | Flurbiprofen (S,R)-, (S)-, (R)- |
| anti-inflammatory | Cyclooxygenase inhibitor | (used as test/marker substrate) |
| Analgesic, antipyretic, | NSAID, Propionic acid deriv., | Ibuprofen (S,R)- |
| anti-inflammatory | Cyclooxygenase inhibitor | |
| Analgesic, antipyretic, | NSAID, Indole acetic acid deriv., | Indomethacin |
| anti-inflammatory | Cyclooxygenase inhibitor | |
| Analgesic, antipyretic, | NSAID, Oxicam Cyclooxygenase | Lornoxicam |
| anti-inflammatory | inhibitor | |
| Analgesic, antipyretic, | NSAID, Anthranilic acid deriv., | Mefenamic acid |
| anti-inflammatory | Cyclooxygenase inhibitor | |
| Analgesic, antipyretic, | NSAID, Oxicam, Cyclooxygenase | Meloxicam |
| anti-inflammatory | inhibitor | |
| Analgesic, antipyretic, | Analgesic, Opioid, | Methadone, (S)- and (R)- |
| anti-inflammatory | Diphenylheptane deriv. | |
| Analgesic, antipyretic, | NSAID, Propionic acid deriv., | Naproxen (S,R)-, (S)-, (R) |
| anti-inflammatory | Cyclooxygenase inhibitor | (used as test/marker substrate) |
| Anagesic Antipyretic, | Analgesic, Antipyretic, p- | Phenacetin |
| anti-inflammatory | Aminophenol | |
| Analgesic, antipyretic, | NSAID, Pyrazolone, | Phenylbutazone |
| anti-inflammatory | Cyclooxygenase inhibitor | |
| Analgesic, antipyretic, | NSAID, Oxicam, Cyclooxygenase | Piroxicam (used as test/marker |
| anti-inflammatory | inhibitor | substrate |
| Analgesic, antipyretic, | NSAID, Propionic acid deriv. | S-2-[4-(3-Methyl-2- |
| anti-inflammatory | | thienyl)phenyl]propionic acid |
| | | (S-MTPPA) |
| Analgesic, antipyretic, | NSAID, Propionic acid deriv., | Suprofen |
| anti-inflammatory | Cyclooxygenase inhibitor | |
| Analgesic, antipyretic, | NSAID, Oxicam, Cyclooxygenase | Tenoxicam |
| anti-inflammatory | inhibitor | |
| Antibacterial | Antileprotic | dapsone |
| Antibacterial | Sulphonamide | sulfadiazine |
| Antibacterial | Sulphonamide | Sulfamethoxazole |
| Antibacterial | Sulphonamide | Sulfamethoxazole |
| Antibacterial | Diaminopyrimidine | Trimethoprim |
| Antidepressant | Tricyclic; P-Glycoprotein (P-gp) | Amitriptyline |
| | weak inhibitor | |
| Antidepressant | Monoamine oxidase type B | Deprenyl (Selegiline) |
| | (MAO-B) inhibitor | |
| Antidepressant | Selective serotonin reuptake | Fluoxetine rac., (S)-, (R)- |
| | inhibitor, SSRI | |
| Antidepressant | Selective alfa2-adrenoreceptor | Mirtazapine |
| | antogonist, Piperazinoazepine | |
| Antidepressant | Selective serotonin reuptake | Sertraline |
| | inhibitor, SSRI | |
| Antidepressant | Serotonin and noradrenaline | Venlafaxine |
| | (norepinefrine) reuptake inhibitor, | |
| | Phenylethylamine | |
| Antidiabetic | Thiazolidinedione | Rosiglitazone |
| Antidiabetic | Sulphonylurea; P-Glycoprotein | Tolbutamide (used as |
| | (P-gp) weak inhibitor | test/marker substrate) |
| Antidiabetic | Thiazolidinedione | Troglitazone |
| Antiepiletic | Hydantoin | Mephenytoin (S)- |
| Antiepiletic | Barbiturate | Phenobarbital |
| Antiepileptic | Hydantoin | Phenytoin |
| Antiepileptic | Oxazolidinedione | Trimethadione (Troxidone) |
| Antiepileptic | Valproate | Valproic acid |
| Antigout | Uricosuric | Sulfinpyrazone sulfide |
| Antihistamine | Piperazine | Cinnarizine |
| (Histamine H1-receptor | | |
| antagonist) | | |
| Antihistamine | Piperazine | Flunarizine |
| (Histamine H1-receptor | | |
| antagonist) | | |
| Antimalarial | Naphthoquinone | 58C80 |
| Antimalarial | Artemisinin derivative | Artelinic acid |
| Antimalarial | Artemisinin derivative | Artelinic acid |
| Antimalarial | Biguanide | Proguanil |
| Antimuscarinic | Tertiary amine | Tolterodine |
| Antineoplastic | Alkylating, oxazaphosphorine | Cyclophosphamide |
| Antineoplastic | Alkaloid | Ellipticine |
| Antineoplastic | Alkylating, oxazaphosphorine | Ifosfamide |
| Antineoplastic | Antiestrogen, nonsteroidal | Tamoxifen |
| Antineoplastic | Selective retinoid X receptor | Targretin (LGD1069) |
| | modulator | |
| Antineoplastic | Nitrosourea | Tauromustine |
| Antipsychotic | Dopamine D2, serotonin2 (5- | Clozapine |
| | HT2) and 5-HT1C receptor | |
| | antagonist, Dibenzodiazepine | |
| Antipsychotic | Phenothiazine | Perazine |
| Antipsychotic | Phenothiazine; P-Glycoprotein | Perphenazine |
| | (P-gp) inhibitor | |
| Antiviral | HIV protease inhibitor; P- | Amprenavir |
| | Glycoprotein (P-gp) | |
| | substrate/inducer | |
| Antiviral | HIV protease inhibitor; P- | Nelfinavir mesylate |
| | Glycoprotein (P-gp) | |
| | substrate/inhibitor/inducer | |
| Antiviral | HIV-1 non-nucleoside reverse | Nevirapine |
| | transcriptase inhibitor | |
| Antiviral | Nucleoside reverse transcriptase | Zidovudine (Azidothymidine, |
| | inhibitor | AZT) |
| Anxiolytic, sedative, | Benzodiazepine | Desmethyladinazolam, N- |
| hypnotic | | |
| Anxiolytic, sedative, | Benzodiazepine | Diazepam |
| hypnotic | | |
| Anxiolytic, sedative, | Benzodiazepine | Flunitrazepam |
| hypnotic | | |
| Anxiolytic, sedative, | Barbiturate | Hexobarbital |
| hypnotic | | |
| Anxiolytic, sedative, | Benzodiazepine | Temazepam |
| hypnotic | | |
| Anxiolytic, sedative, | Insomnia agent, Imidazopyridine | Zolpidem |
| hypnotic | | |
| Bronchodilators and | 5-Lipoxygenase inhibitor | ABT-761 and ABT-438 |
| Anti-asthma | | (metabolite, N-hydroxy) |
| Bronchodilators and | Leucotriene D4 (LTD4) receptor | Zafirlukast |
| Anti-asthma | selective antagonist | |
| Cardiovascular | Thromboxane A2 (TXA2) | (+)-(S)-145 and derivatives |
| | receptor antagonist | |
| Cardiovascular | Anticoagulant | Acenocoumarol (R/S)- |
| Cardiovascular | beta-Adrenoceptor blocking | Bufuralol |
| | agent (Beta blocker) | |
| Cardiovascular | Angiotensin II receptor antagonist | Candesartan |
| | (sartan) | |
| Cardiovascular | beta-Adrenoceptor blocking | Carvedilol (S)- and (R)- |
| | agent (Beta blocker); P- | |
| | Glcyoprotein (P-gp) inhibitor | |
| Cardiovascular | Anticoagulant | Dicoumarol |
| Cardiovascular | Calcium-channel blocker, | Diltiazem |
| | benzothiazepine; P-Glycoprotein | |
| | (P-gp) substrate/weak inhibitor | |
| Cardiovascular | Carbonic anhydrase inhibitor | Dorzolamide |
| Cardiovascular | Angiotensin II receptor antagonist | Irbesartan |
| Cardiovascular | Angiotensin II receptor antagonist | Losartan |
| | (sartan); P-Glycoprotein (P-gp) | |
| | substrate | |
| Cardiovascular | Anticoagulant | Phenprocoumon |
| Cardiovascular | Antiarrhythmic, Cinchona | Quinidine |
| | akaloid, 4-Methanolquinoline; P- | |
| | Glycoprotein (P-gp) | |
| | inhibitor/substrate | |
| Cardiovascular | Antiplatelet, Thromboxane A2 | Seratrodast |
| | (TXA2) receptor antagonist | |
| Cardiovascular | Diuretic and uricosuric | Tienilic acid |
| Cardiovascular | Diuretic, loop | Torasemide (Torsemide) |
| Cardiovascular | Calcium-channel blocker, | Verapamil, rac, (R)-, (S)- |
| | phenylalkylamine; P-Glycoprotein | |
| | (P-gp) inhibitor | |
| Cardiovascular | Anticoagulant | Warfarin (S,R)-, (S)- and (R)- |
| Cough suppressant | Centrally acting | Dextromethorphan |
| Dermatological agent | Retinoic acid receptor modulator | Retinoic acid, 9-cis- (Panretin) |
| Dermatological agent | Retinoic acid receptor modulator | Retinoic acid, all trans- |
| | | (Tretinoin) |
| Erectile dysfunction | cGMP-specific | Sildenafil (Viagra) |
| | phosphodiesterase type 5 | |
| | inhibitor | |
| Gastro-intestinal | 5-HT3-receptor antagonist | Alosetron |
| Gastro-intestinal | Serotonin-5-HT4-receptor | Cisapride |
| | agonist, piperidinyl benzamide | |
| Gastro-intestinal | 5-HT3-receptor antagonist | Doiasetron |
| Gastro-intestinal | Benzimidazole (Omeprazole | H 259/31 |
| | deriv.) | |
| Gastro-intestinal | Proton pump inhibitor, | Lansoprazole |
| | benzimidazole | |
| Gastro-intestinal | Proton pump inhibitor, | Omeprazole (S)- |
| | pyridinylsulfinylbenzimidazole; P- | (Esomeprazole) |
| | Glycoprotein (P-gp) inhibitor | |
| Gastro-intestinal | 5-HT3-receptor antagonist | Ondansetron |
| Gastro-intestinal | Natural compound, Antiemetic, | Tetrahydrocannabinol THC, |
| | Marijuana (Cannabis) const. | delta1- (delta9-) |
| Gastro-intestinal | 5-HT3-receptor antagonist | Tropisetron |
| Gastro-intestinal | 5-Lipoxygenase inhibitor | Zileuton |
| Gastro-intestinal | 5-Lipoxygenase inhibitor | Zileuton, N-dehydroxy |
| | | metabolite |
| General anesthetic | Halogenated | Halothane |
| General anesthetic | NMDA receptor antagonist, | Ketamine (R)-, (S)- |
| | phencyclidine deriv. | |
| General anesthetic | Di-isopropylphenol | Propofol |
| General anesthetic | Thiobarbiturate | Thiamylal (S)- and (R)- |
| Lipid regulating | HMB-CoA reductase inhibitor | Fluvastatin |
| | (Statin); P-Glycoprotein (P-gp) | |
| | substrate | |
| Lipid regulating | HMG-CoA reductase inhibitor | NK-104 |
| Local anesthetic | Amide type | Lidocaine (Lignocaine) |
| Other chemical | Polycyclic aromatic hydrocarbon | (+)- and (−)-7,8-Dihydroxy-7,8-Dihydroxy-7,8- |
| | (PAH) | dihydro-benzo[a]pyrene, (+)- |
| | | and (−)-B[a]P-7,8-diol |
| Other chemical | Polycyclic aromatic hydrocarbon | (+)-(11S, 12S)- and (−)- |
| | (PAH) | (11R, 12R)- |
| | | Dihydroxydibenzo[a,l]pyrene |
| | | (DB[a,l]P-11,12-diol) |
| Other chemical | Polycyclic aromatic hydrocarbon | 1,2-Dihydroxy-1,2-dihydro-5,6- |
| | (PAH) | dimethylchrysene (5,6- |
| | | Dimethylchrysene-1,2-diol) |
| Other chemical | Polycyclic aromatic hydrocarbon | 11,12-Dihydroxy-11,12- |
| | (PAH) | dihydrobenzo[g]chrysene |
| | | (Benzo[g]chrysene-11,12-diol, |
| | | B[g]C-11,12-diol) |
| Other chemical | Heterocyclic amine | 2-Amino-1-methyl-6- |
| | | phenylimidazo[4,5b]pyridine, |
| | | PhIP |
| Other chemical | Heterocyclic, aromatic amine | 2-Amino-3,4- |
| | | dimethylimidazo[4,5- |
| | | f]quinoline, MelQ |
| Other chemical | Heterocyclic, aromatic amine | 2-Amino-3-methylimidazo[4,5- |
| | | f]quinoline, IQ |
| Other chemical | Aromatic amine, Arylamine | 2-Amino-6-methyldipyrido[1,2- |
| | | a:3,2′-d]-imidazole, Glu-P-1 |
| Other chemical | Aromatic amine, Arylamine | 2-Aminoanthracene, 2-AA |
| Other chemical | Aromatic amine, Arylamine | 2-Aminofluorene, 2-AF |
| Other chemical | 2-Aroylthiophene | 2-Aroylthiophenes (beraing |
| | | negative charge) |
| Other chemical | Polycyclic aromatic hydrocarbon | 3,4-Dihydroxy-3,4- |
| | (PAH) | dihydrobenzo[c]phenanthrene |
| | | (Benzo[c]phenanthrene-3,4- |
| | | diol, B[c]P-3,4-diol) |
| Other chemical | Heterocyclic, aromatic amine | 3-Amino-1,4-dimethyl-5H- |
| | | pyrido[4,3-b]indole (Trp-P-1) |
| Other chemical | Heterocyclic, aromatic amine | 3-Amino-1-methyl-5H- |
| | | pryrido[4,3-b]indole (Trp-P-2) |
| Other chemical | Alkyloxycoumarin | 3-Cyano-7-ethoxycoumarin |
| Other chemical | Alkyloxyfluorescein | 3-O-methylfluorescein (used |
| | | as test/marker substrate) |
| Other chemical | Polycyclic aromatic hydrocarbon | 7,12- |
| | (PAH) | Dimethylbenz[8a]anthracene |
| | | (7,12-DMBA) |
| Other chemical | Polycyclic aromatic hydrocarbon | 7,12- |
| | (PAH) | Dimethylbenz[a]anthracene- |
| | | 3,4-diol (7,12-DMBA-3,4-diol) |
| Other chemical | Polycyclic aromatic hydrocarbon | 7,8-Dihydroxy-7,8- |
| | (PAH) | dihydrobenzo[a]pyrene, B[a]P- |
| | | 7,8-diol |
| Other chemical | Alkyloxycoumarin | 7-Benzyloxy-4- |
| | | trifluoromethylcoumarin, BFC |
| Other chemical | Alkyloxycoumarin | 7-Ethoxy-4- |
| | | trifluoromethylcoumarin |
| Other chemical | Alkyloxycoumarin | 7-Ethoxycoumarin |
| Other chemical | Polycyclic aromatic hydrocarbon | Benz[a]anthracene (1,2- |
| | (PAH) | Benzanthracene) |
| Other chemical | Polycyclic aromatic hydrocarbon | Benz[a]anthracene-3,4-diol |
| | (PAH) | |
| Other chemical | Polycyclic aromatic hydrocarbon | Benzo[a]pyrene, B[a]P |
| | (PAH) | |
| Other chemical | Polycyclic aromatic hydrocarbon | Benzo[b]fluoranthene-9,10-diol |
| | (PAH) | (B[b]F-9,10-diol) |
| Other chemical | Endocrine disruptor, estrogen | Bisphenol A |
| | activity | |
| Other chemical | Unclassified | Butadiene monoxide |
| | | (Epoxybutene |
| Other chemical | Polycyclic aromatic hydrocarbon | Chrysene-1,2-diol |
| | (PAH) | |
| Other chemical | Polycyclic aromatic hydrocarbon | Dibenzo[a,h]anthracene |
| | (PAH) | |
| Other chemical | Polycyclic aromatic hydrocarbon | Dibenzo[a,l]pyrene (DB[a,l]P) |
| | (PAH) | |
| Other chemical | Polycyclic aromatic hydrocarbon | Dibenzo[a]pyrene |
| | (PAH) | |
| Other chemical | Alkloxyfluorescein | Dibenzylfluorescein, DBF |
| | | (suggested as test/marker |
| | | substrate) |
| Other chemical | Alkyloxyfluorescein | Diethoxy(−5/−6)chloromethyl |
| | | fluorescein (DECMF) |
| Other chemical | Polycyclic aromatic hydrocarbon | Naphthalene |
| | (PAH) | |
| Other chemical | Nonionic phenolic detergent | Triton N-101 |
| Pesticide | Insecticide, Chlorinated | Methoxychlor |
| Physiological | Steroid | 5alpha-Androstane-3alpha, |
| compound | | 17beta-diol |
| Physiological | Fatty acid | Arachidonic acid |
| compound | | |
| Physiological | Fatty acid | Linoleic acid |
| compound | | |
| Physiological | Hormone, Methoxytryptamine | Melatonin |
| compound | | |
| Sex hormone | Progestagen | Desogestrel |
| Sex hormone | Estrogen | Estradiol, 17beta- |
| Sex hormone | Estrogen | Estradiol, 3-methyl ether |
| Sex hormone | Estrogen | Estrone |
| Sex hormone | Estrogen, synthetic, | Mestranol |
| | contraceptive | |
| Sex hormone | Progestagen; P-Glycoprotein (P- | Progesterone |
| | gp) inhibitor | |
| Sex hormone | Androgen and anabolic | Testosterone |
| Supplementary drugs | Natural compound, Monoterpene | 1,8-Cineole (Eucalyptol) |
| and other substances | cyclic ether, Eucalyptus | |
| | polybractea const. | |
| Supplementary drugs | Schizandrin C deriv., used as | DDB |
| and other substances | hepatoprotective | |
| Supplementary drugs | Natural compound, Garlic oil | Diallyl disulfide (DADS) |
| and other substances | component, Organosulfur | |
| Supplementary drugs | Natural compound, Alkaloid | Nicotine |
| and other substances | | |
| Supplementary drugs | Natural compound, Mycotoxin | Ochratoxin A |
| and other substances | | |
| Supplementary drugs | Antialcoholic | S-methyl N,N, |
| and other substances | | diethylthiolcarbamate (DETC- |
| | | ME) |
| Supptementary drugs | Natural compound, Marijuana | Tetrahydrocannabinol THC, |
| and other substances | Cannabis) const. | 7alpha-hydroxy-delta8- |
| Xanthine | Bronchodilator | Theophylline |
| Xanthine, Food | CNS stimulant; P-Glycoprotein | Caffeine |
| component | (P-gp) weak inhibitor | |

Induction and Inhibition

CYP 2C9 is inhibited by fluconazole, metronidazole, miconazole, ketoconazole, itaconazole, ritonavir, clopidrogel, amiodarone, fluvoxamine, sulfamthoxoazole, fluvastatin and fluoxetine. It is induced by rifampin and rifabutin. The ability to quickly and easily determine an individual's CYP 2C9-specific phenotype allows a physician to determine the phenotypic status of an individual and make a corresponding determination about the type and extent of treatment most suitable at a given time. The present invention provides a reliable method of identifying a suitable drug compatible with an individual's phenotype, as well as a method of individualizing therapy with a specific drug(s) with respect to dosage, duration etc. based thereon.

In accordance with an embodiment of the present invention there is provided a phenotypic determinant specific for CYP 2C9 metabolism. This phenotypic determinant provides an indication of an individual's CYP 2C9 phenotype. Furthermore, the phenotypic determinant may be used to provide a drug response profile for the individual specific to drug(s) known to be metabolized by the CYP 2C9 pathway.

Inter Ethnic Differences

The CYP 2C9 genotypes demonstrate marked inter-ethnic variability. The CYP2C9*2 is absent from Chinese, Taiwanese and present in only 1% of African American populations, but accounts for 19.2% of the British population and 8% of Caucasians. CYP2C9*3 is more rare is and is present in 6% of Caucasian, 2% of Chinese, 2.6% of Taiwanese and 0.5% of African-American populations.

It is reasonable that, in drug metabolism studies, each ethnic group can be studied separately for evidence of polymorphism and its antimode should not be extrapolated from one ethnic population to another. Furthermore, this inter-ethnic variability provides a clear indication that efforts to individualization treatments should be made to overcome the risks and inefficiencies currently experienced with standardized dosing.

S-warfarin

As an example, the benefit of CYP 2C9 metabolic phenotyping in drug dosing is evident in the case of S-warfarin. S-warfarin is an anticoagulant drug. Studies have demonstrated that the presence of either CYP2C9*2 or CYP2C9*3 haplotypes-mutants results in a decrease in the dose necessary to acquire target anticoagulation intensity. In addition, these individuals also suffered from an increased incidence of bleeding complications. Therefore, the CYP 2C9 gene variants modulate the anticoagulant effect of the dose of warfarin prescribed. Clearly, the ability to readily determine the presence of such mutant alleles prior to treatment would prove beneficial as a compatible dosage of S-warfarin could then be determined. Thus alleviating or eliminating the occurrence of adverse side effects.

For these reasons, the utility of a reliable test for CYP 2C9 is evident. In particular, an accurate and convenient clinical assay would allow physicians to quickly identify safe and effective treatment regimes for individuals on an individual basis. In addition, the present invention provides a means to determine the efficiency of an individual's CYP 2C9 metabolism before prescribing a standard treatment. In doing so, a standard s treatment may then be tailored to provide an individualized treatment that will correspond with an individual's CYP 2C9 phenotype.

Direct Phenotypic Determinants of CYP 2C9

Different substrates (or probe substrates) such as ibuprofen, losartan, tolbutamide, lurbiprofen, diclofenac, phenytoin & warfarin can be used to determine a CYP 2C9 phenotype according to the present invention. (s)-ibuprofen is exemplified as a probe substrate, without limitation, in accordance the present invention.

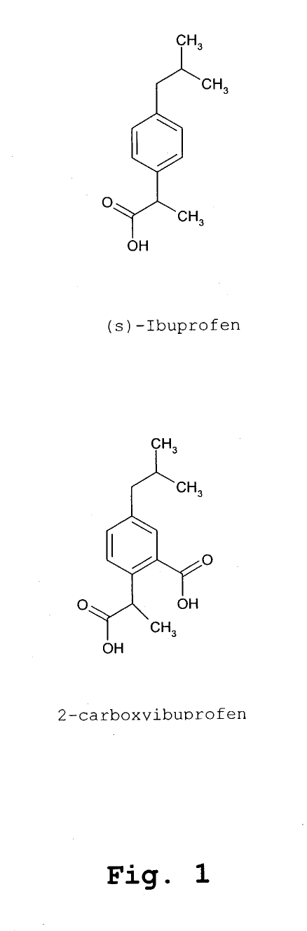

According to one embodiment of the present invention, the ratio of (s)-ibuprofen and its carboxylated metabolite, 2-carboxyibuprofen in a urine sample may be used to provide a phenotypic determinant corresponding to an individual's CYP 2C9 phenotype. This metabolite is used as a quantitative marker in the determination of a CYP 2C9 phenotype on the basis of the use of the preferred probe substrate (s)-ibuprofen. The structures of (s)-ibuprofen and its metabolite 2-carboxyibuprofen are illustrated in FIG. 1. However, it is fully contemplated that the present invention is not limited in any respect thereto. In fact, due to the nature of the substrate specific alterations caused by the individual CYP 2C9 mutations, multiple probe substrates may be employed for a phenotypic determination of CYP 2C9.

The molar ratio of (s)-ibuprofen and its 2-carboxyibuprofen metabolite, used to determine the CYP 2C9 phenotype of the individual, is as follows:

$$\frac{( s ) - ibuprofen}{2 - carboxyibuprofen}$$

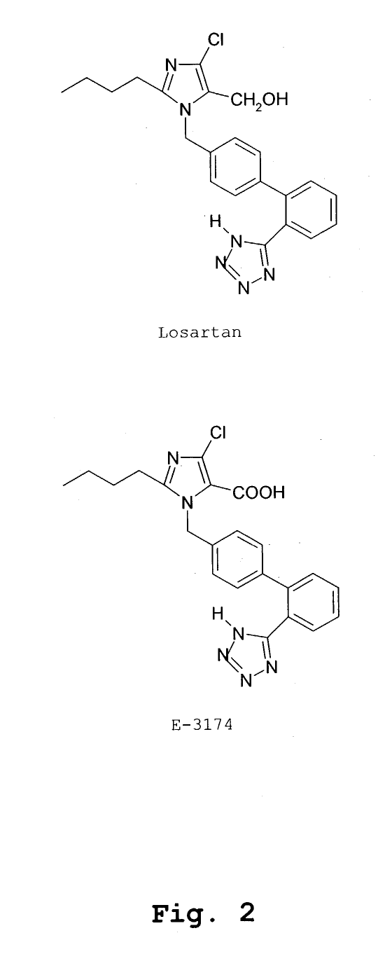

According to another embodiment of the present invention, the ratio of losartan and its metabolite E-3174 in a urine sample may be used to provide a phenotypic determinant corresponding to an individual's CYP 2C9 phenotype. This metabolite is used as a quantitative marker in the determination of a CYP 2C9 phenotype on the basis of the use of the preferred probe substrate losartan. The structures of losartan and its metabolite E-3174 are illustrated in FIG. 2. However, it is fully contemplated that the present invention is not limited in any respect thereto. In fact, due to the nature of the substrate specific alterations caused by the individual CYP 2C9 mutations, multiple probe substrates may be employed for a phenotypic determination of CYP 2C9.

The molar ratio of losartan and its metabolite E-3174, used to determine the CYP 2C9 phenotype of the individual, is as follows:

$$\frac{Losartan}{E - 3174}$$

Enzyme linked immunosorbent assays (ELISA) have been successfully applied in the determination of low amounts of drugs and other antigenic compounds in plasma and urine samples and are simple to carry out. An ELISA for N-acetyltransferase-2 (NAT2) phenotyping using caffeine as a probe substrate has also been developed and validated (Wong, P., Leyland-Jones, B., and Wainer, I. W. (1995) J. Pharm. Biomed. Anal. 13: 1079-1086); (Leyland-Jones et al. (1999) Amer. Assoc. Cancer Res. 40: Abstract 356). The ELISA for NAT2 phenotyping is simpler to carry out than the HPLC and CE.

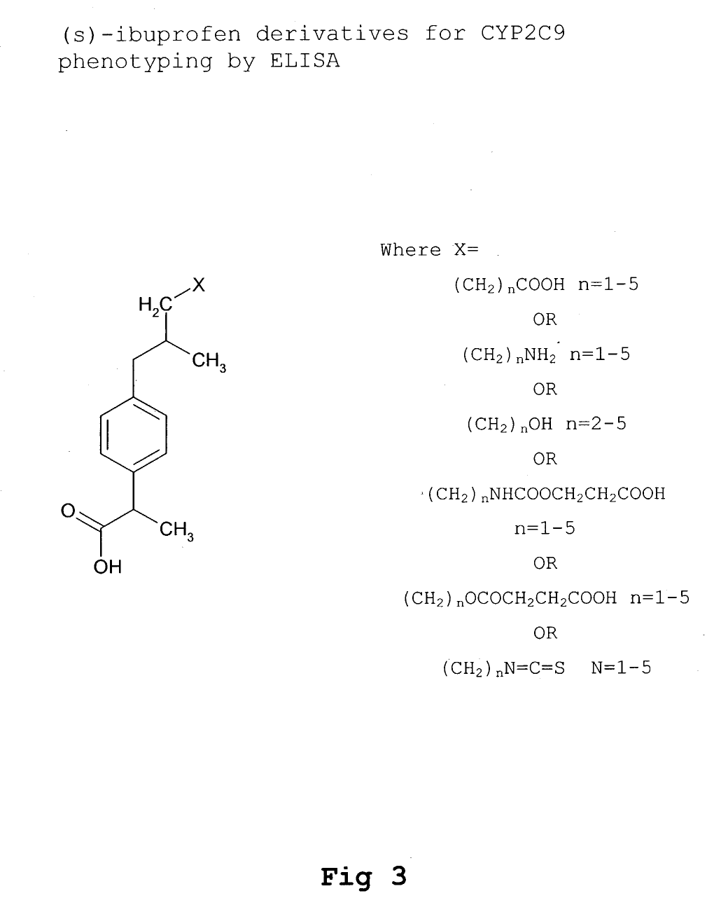

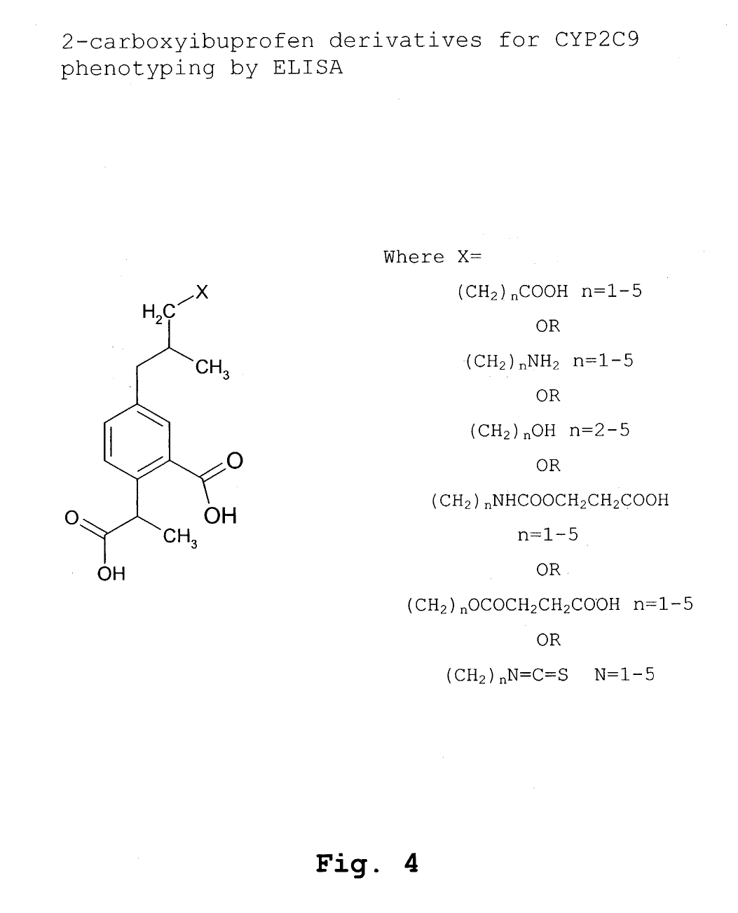

In developing the antigen enzyme linked immunosorbent assay (ELISA) of the present invention, antibodies to (s)-ibuprofen and 2-carboxyibuprofen have been developed to measure the molar ratio of these compounds in urine samples collected from an individual after (s)-ibuprofen consumption. The antibodies of the present invention can be polyclonal or monoclonal antibodies raised against derivatives of (s)-ibuprofen and 2-carboxyibuprofen, as exemplified in FIGS. 3 and 4, respectively. Based on the development of these derivatives and subsequently derived antibodies, the ability to determine the molar ratio of (s)-ibuprofen and 2-carboxyibuprofen, in accordance with the present invention, was achieved.

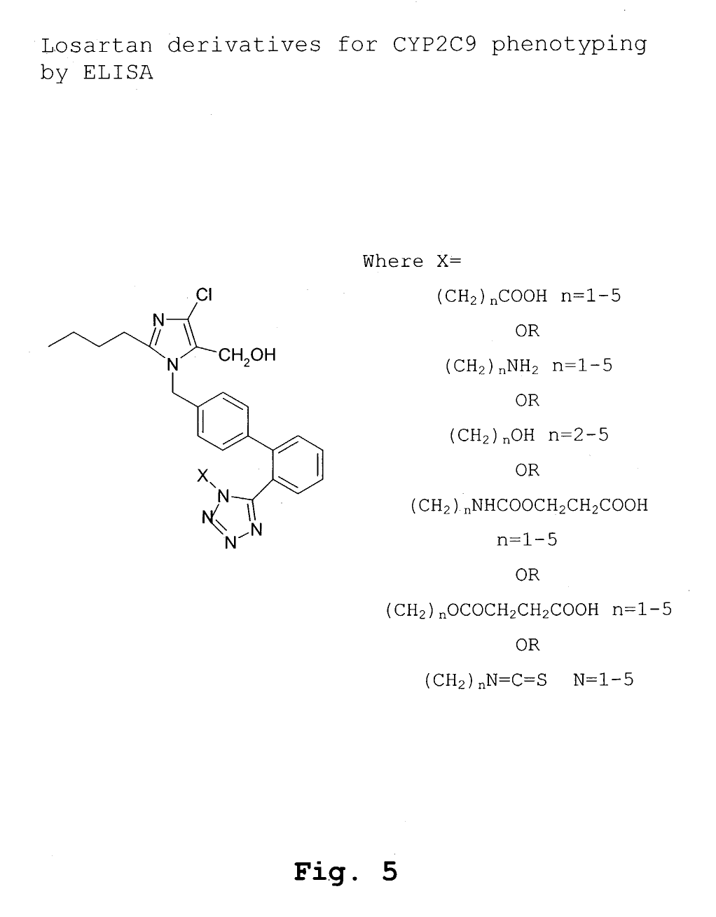

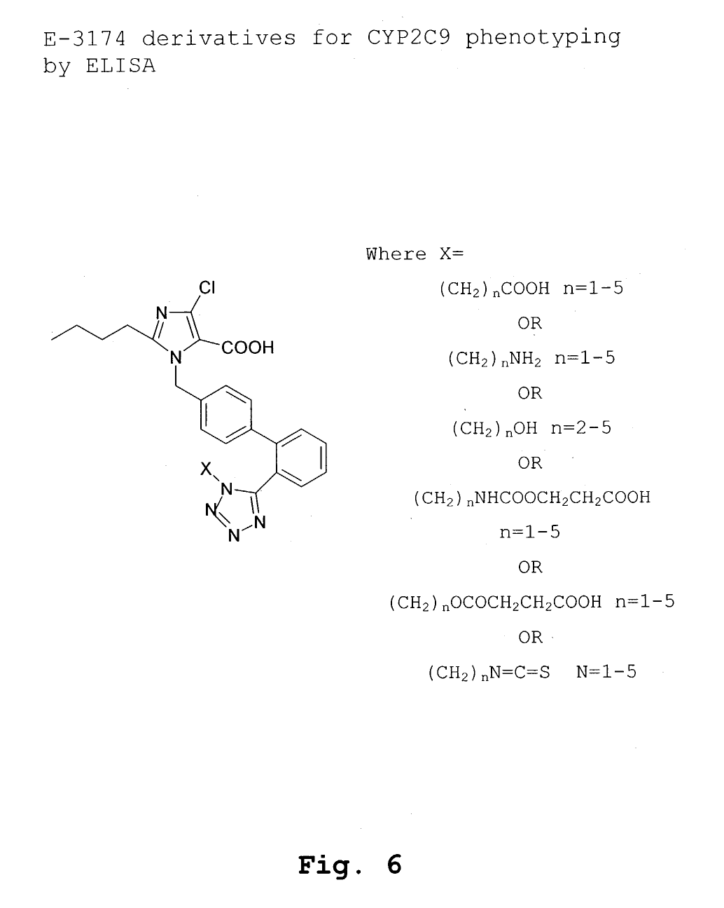

In accordance with an embodiment of the present invention the antibodies of the present invention can be polyclonal or monoclonal antibodies raised against derivatives of losartan and E-3174, as exemplified in FIGS. 5 and 6, respectively.

In accordance with an embodiment of the present invention, the ratio of (s)-ibuprofen and 2-carboxyibuprofen in a urine sample may be used to provide a determination of an individual's CYP 2C9 phenotype. These compounds are used as quantitative markers in the determination of a CYP 2C9 phenotype on the basis of the use of the preferred probe substrate (s)-ibuprofen. However, it is fully contemplated that the present invention is not limited in any respect thereto.

In accordance with an embodiment of the present invention, the ratio of losartan and E-3174 in a urine sample may be used to provide a determination of an individual's CYP 2C9 phenotype. These compounds are used as quantitative markers in the determination of a CYP 2C9 phenotype on the basis of the use of the preferred probe substrate losartan. However, it is fully contemplated that the present invention is not limited in any respect thereto.

In accordance with another embodiment of the present invention, a competitive antigen ELISA is provided for determining CYP 2C9 phenotyping using (s)-ibuprofen as the probe substrate. The assay is sensitive, rapid and can be readily carried out on a routine basis by a technician with a minimum of training in a clinical laboratory.

The present invention will be more readily understood by referring to the following Materials and Methods and Examples which are given to illustrate the invention rather than to limit its scope.

### MATERIALS AND METHODS

Materials

Horse radish peroxidase is purchased from Boehringer Mannheim (Montreal, Que., Canada); ELISA plates (96-well Easy Wash™ modified flat bottom, high binding); Corning glass wares, (Corning, N.Y., USA) and Falcon 96-well microtest tissue culture plate, no. 3072 (Beckton Dickinson Labware, Franklin, N.J., USA) are purchased from Fisher (Montreal, Que., Canada); alkaline phosphatase conjugated to goat anti-rabbit IgGs, Keyhole limpet hemocyanin (KLH) is from Pierce Chemical Co. (Rockford, Ill., USA); acetic anhydride, acetonitrile HPLC grade, benzylurea, bovine serum albumin (Cat. No. A-3803), N-bromosuccinimide,; 1-ethyl-3-(3-dimethylaminopropyl) carbodiimide hydrochloride solution (EDAC), diethanolamine, Freund's adjuvant (complete and incomplete), glutaraldehyde (50% v/v), p-nitrophenolphosphate disodium salt, palladium, 10 wt. % (dry basis) on activated carbon, o-phenylenediamine hydrochloride, polyoxyethylene sorbitan monolaurate (Tween™ 20), porcine skin gelatin, protein A-Sepharose 4B, Sephadex™ G25 fine, sodium hydride, tributylamine, Tween™ 20, are purchased from Sigma-Aldrich (St-Louis, Mo., USA); Silica gel particle size 0.040-0.063 mm (230-400 mesh) ASTM Emerck Darmstadt, Germany is purchased from VWR (Montreal, Que., Canada). Dioxane is dried by refluxing over calcium hydride for 4 hours and distilled before use. Other reagents are ACS grade.

Synthesis of Derivatives of (s)-ibuprofen and 2-carboxyibuprofen

The (s)-ibuprofen and 2-carboxyibuprofen derivatives may include, without limitation those illustrated in FIGS. 3 and 4.

Conjugation of Haptens to Bovine Serum Albumin (BSA) and Keyhole Limpet Hemocyanin

(s)-Ibuprofen-BSA and 2-carboxyibuprofen-BSA conjugates are prepared by a procedure similar to that of Rojo et al. (Rojo et al. (1986) J Immunol. 137: 904-910). In a 25 mL erlenmeyer flask 15 mg of BSA is dissolved in 6 mL of a (s)-ibuprofen derivative (or 2-carboxyibuprofen derivative) solution (1.25 μmoles/mL of water) followed by the addition of 1.43 mL of an EDAC solution (10 mg/mL of water). The solution is stirred overnight at room temperature and dialyzed against 500 mL water at room temperature for 48 hours with two changes per day of the water. The conjugates are stored as 0.5 mL-aliquots at −20° C. In addition, the conjugates may be prepared by the method of Peskar et al. (Peskar (1972) Eur. J. Biochem. 26: 191-195). In a 5 mL round bottom flask 7.5 mg of (s)-ibuprofen derivative (or 2-carboxyibuprofen derivative) (0.03 mmole) is placed and is dissolved with 1 mL of a 0.1M Na₂HPO₄—NaH₂PO₄ buffer, pH 7.0. A volume of 500 μL of a 0.021 M glutaraldehyde solution (42.5 μL 50% glutaraldehyde (v/v) per 10 mL of water) is added to the stirred solution. After stirring for 2 hours, 100 μL of a 1M lysine solution in 0.1M Na₂HPO₄—NaH₂PO₄ buffer, pH 7.0 is is added. The solution is stirred for one hour and dialyzed against 250 mL of a 150 mM NaCl, 5 mM Na₂HPO₄—NaH₂PO₄ buffer, pH 7.0 for 48 hours with 2-3 changes per day of the buffer. Solution BSA conjugates are stored as 0.5 mL aliquots at −20° C.

(s)-Ibuprofen-KLH and 2-carboxyibuprofen-KLH conjugates are prepared as follows. First, 20 mg of lyophilized powder of KLH is dissolved with 2 mL of a 0.9 M NaCl solution and dialyzed against 100 mL of water for 10 hours with 2 changes of the water. To 1.1 mL KLH solution (˜10 mg/mL) in a 25 mL erlenmeyer flask, 0.8 mL of the (s)-ibuprofen derivative (or 2-carboxyibuprofen derivative) (2.5 μmol/mL in 0.9 M NaCl). 2 mL of an EDAC solution (10 mg/mL in 0.9 M NaCl), and 1.8 mL 0.9 M NaCl solution are successively added to the derivative solution. The solution is stirred overnight (20 hours) at room temperature. The solution is dialyzed against 250 mL of a 0.9 M NaCl solution for 48 hours with 2-3 changes of the solution per day. (s)-ibuprofen-KLH and 2-carboxyibuprofen-KLH solutions are stored as 0.5 mL aliquots at −20° C. In addition, the conjugates may be prepared according to a method similar to that of Peskar et al. (Peskar (1972) Eur. J. Biochem. 26: 191-195). First, 20 mg of lyophilized powder of KLH is dissolved with 2 mL of a 0.9 M NaCl solution and dialyzed against 100 mL of water for 10 hours with 2 changes of the water. Approximately 0.03 mmole of (s)-ibuprofen or 2-carboxyibuprofen is placed in a 5 mL round bottom flask and is dissolved with 1 mL of the KLH solution. A volume of 500 μL of a 0.021 M glutaraldehyde solution (42.5 μL 50% glutaraldehyde (v/v) per 10 mL of water) is added dropwise to the stirred solution. After stirring for 2 hours, 100 μL of a 1M lysine solution in 0.1M Na₂HPO₄—NaH₂PO₄ buffer, pH 7.0 is added. The solution is stirred for one hour and dialyzed against 250 mL of a 0.9M NaCl, 5 mM Na₂HPO₄—NaH₂PO₄ buffer, pH 7.0 for 48 hours with 2-3 changes per day of the buffer. Solutions of BSA conjugates are stored as 0.5 mL aliquots at −20° C.

Protein Determination

Protein determination was performed according to the method of Lowry et al. as described in Lowry, O. H. et al. (1951) J. Biol. Chem., 193: 265-275, which is herein incorporated by reference.

Solutions

| | |
|---|---|
| Solution A: | 2 g Na₂CO₃ is dissolved in 50 mL water, |
| | 10 mL of 10% SDS and 10 mL 1 N NaOH; bring |
| | to 100 mL volume with water. Freshly |
| | prepared. |
| Solution B: | 1% NaK Tartrate |
| Solution C: | 1% CuSO₄.5H₂O |
| Solution D: | 1 N phenol (freshly prepared): 3 mL Folin |
| | & Ciaocalteu's phenol reagent (2.0 N) and |
| | 3 mL water. |
| Solution E: | 98 mL Solution A, 1 mL Solution B, 1 mL |
| | Solution C. Freshly prepared. |
| BSA: | 1 mg/mL. 0.10 g bovine serum albumin |
| | (fraction vol.)/100 mL water. |

Assay

| Standard curve | Tube # (13 × 100 mm) | | | | | | |
|---|---|---|---|---|---|---|---|
| Solution | 1 | 2 | 3 | 4 | 5 | 6 | 7 |
| BSA (μl) | 0 | 10 | 15 | 20 | 30 | 40 | 50 |
| Water (μl) | 200 | 190 | 185 | 180 | 170 | 160 | 150 |
| Solution E (mL) | 2.0 | 2.0 | 2.0 | 2.0 | 2.0 | 2.0 | 2.0 |

The solutions are vortexed and left for 10 min at room temperature.

| | | | | | | | |
|---|---|---|---|---|---|---|---|
| Solution D (μl) | 200 | 200 | 200 | 200 | 200 | 200 | 200 |

The solutions are vortexed and left at room temperature for 1 hour.

The absorbance is read at 750 nm using water as the blank.

Unknown

| Solution D.F^{a} (in triplicate) | Tube # (13 × 100 mm) | | |
|---|---|---|---|
| Unknown (μl) | x | x | x |
| Water (μl) | y | y | y |
| (x + y = 200 μl) | | | |
| Solution F (mL) | 2.0 | 2.0 | 2.0 |
| The solutions are vortexed and left for 10 min at room temperature. | | | |
| Solution D (μl) | 200 | 200 | 200 |

The solutions are vortexed and left at room temperature for 1 hour.

The absorbance is read at 750 nm using water as the blank. The protein concentration is calculated using the standard curve and taking in to account the D.F. (dilution factor) of the unknown.

a: D.F. (dilution factor): has to be such that the absorbance of the unknown at 750 nm is within the range of absorbance of the standards.

Methods to Determine the Amounts of Moles of (s)-ibuprofen or 2-carboxyibuprofen Incorporated per mg of KLH

This method gives an approximate estimate. It is useful because it allows the determination of whether the coupling proceeded as expected.

A) Solutions

10% sodium dodecyl sulfate (SDS) solution

1% SDS solution

0.5 or 1 mg/mL of (s)-ibuprofen-KLH (or 2-carboxyibuprofen-KLH) in a 1% SDS solution (1 mL)

0.5 or 1 mg/mL KLH in a 1% SDS solution

B) Procedure

The absorbance of the (s)-ibuprofen-KLH conjugate (or 2-carboxyibuprofen-KLH) is measured at the wavelength of absorption maximum of (s)-ibuprofen, with a 1% SDS solution as the blank.

The absorbance of the KLH solution is measured at the wavelength of absorption maximum of (s)-ibuprofen, with a 1% SDS solution as the blank.

The amount of mole of (s)-ibuprofen incorporated per mg of KLH is calculated with the following formula:

$$y = \frac{{A}_{λ max} ( ibuprofen - KLH ) - {A}_{λ max} ( KLH )}{{ℰ}_{λ max} ( ibuprofen ) \times [ KLH ]}$$

where:

y is the amount of mole of (s)-ibuprofen/mg of KLH;

ε_{λmax}((s)-ibuprofen) is the molar extinction coefficient of (s)-ibuprofen at the wavelength of absorption maximum.

Coupling of Haptens to Horse Radish Peroxidase

The (s)-ibuprofen and 2-carboxyibuprofen derivatives (after succinylation with succinic anhydride) are conjugated to horse radish peroxidase (HRP) by the following procedure. In a 5 mL round bottom flask are placed 0.12 mmol of the derivative. Then, 500 μL of dioxane freshly dried over calcium chloride is added. The suspension is stirred and cooled at 10° C. in a water bath using crushed ice. Then, 31 μL isobutylchloroformate (0.24 mmol) (recently opened or purchased) and 114 μL tributylamine (0.47 mmol) are added. The suspension is stirred for 30 min at 10° C. While stirring, 13 mg of horse radish peroxidase (HRP) is dissolved in 2 mL of water and the solution is cooled at 4° C. on crushed ice. After the 30 min of stirring, 100 μL of a 1N NaOH solution (freshly prepared) at 4° C. is added to the HRP solution and the alkaline HRP solution is poured at once in the 5 mL flask. The suspension is stirred for 4 hours at 10-12° C. The free derivative is separated from the HRP conjugate by filtration on a Sephadex G-25™ fine column (1.6×30 cm) equilibrated and eluted with 0.1 M sodium phosphate buffer, pH 7.0. The fractions of 1.0-1.2 mL are collected manually or with a fraction collector. During elution two bands may be observed: the HRP conjugate and a light yellow band behind the HRP conjugate. The HRP conjugate band is eluted between fractions 11-16. The fractions containing the HRP conjugate are pooled in a 15 mL tissue culture with a screw cap. The HRP conjugate concentration is determined at 403 nm after diluting an aliquot (usually 50 μL+650 μL of buffer).

[HRP conjugate] (mg/mL)=A₄₀₃×0.4×D.F.

After the reaction is complete, 5 μL of a 4% thiomersal solution is added per mL of (s)-ibuprofen-HRP or 2-carboxyibuprofen-HRP conjugate solution. The conjugates are stored at 4° C.

Antibody Production

Four mature females New Zealand white rabbits (Charles River Canada, St-Constant, Que., Canada) are used for antibody production. An isotonic saline solution (0.6 mL) containing 240 μg of KLH conjugated antigen is emulsified with 0.6 mL of a complete Freund's adjuvant. Then, 0.5 mL of the emulsion (100 μg of antigen) is injected per rabbit intramuscularly or subcutaneously. Rabbits are subsequently boosted at intervals of three weeks with 50 μg of antigen emulsified in incomplete Freund's adjuvant. Blood is collected without anticoagulant in a vacutainer tube by venipuncture of the ear 10-14 days after boosting and kept at 4° C. After clotting, centrifugation at 4° C., sodium azide is added to the antisera to a final concentration of 0.001% (1 μL of a 1% sodium azide solution per mL of antisera). Antisera is stored as 0.5 mL aliquots at −20° C.

Antiserum Titers

The wells of a microtiter plate are coated with 10 μg mL⁻¹ of bovine serum albumin-(s)-ibuprofen(or R-mephenytoin) conjugate in 100 mM sodium carbonate buffer, pH 9.6) overnight at 4° C. (150 μL/well). The wells are then washed three times with TPBS (phosphate buffered saline containing 0.05% Tween™ 20) using a Nunc Immuno Wash 12 autoclavable. Unoccupied sites are blocked by an incubation with 150 μL/well of TPBS containing 0.05% porcine gelatin for 2 h at room temperature. The wells are washed three times with TPBS and 150 μL of antiserum diluted in TPBS is added. After 2 h at room temperature, the wells are washed three times with TPBS, and 100 μL of goat anti-rabbit IgGs-alkaline phosphatase conjugate diluted in PBS containing 1% BSA are added. After 1 h at room temperature, the wells are washed three times with TPBS and three times with water. To the wells are added 150 μL of a solution containing MgCl₂ (0.5 mM) and p-nitrophenol phosphate (3.85 mM) in diethanolamine buffer (10 mM, pH 9.8). After 30 min at room temperature, the absorbency is read at 405 nm with a microplate reader. The antibody titer is defined as the dilution required to change the absorbance by one unit (1 au).

Isolation of IgG Antibodies

Rabbit IgG antibodies against KLH conjugates are purified by affinity chromatography on a Protein A-Sepharose 4B column as follows. A 0.9×15 cm Pharmacia chromatographic column is packed with Protein A-Sepharose 4B suspension to a volume of 1 mL. The column is washed generously with a 0.01 M Na₂HPO₄—NaH₂PO₄ buffer, pH 8.0 containing 0.15 M NaCl (PBS) and then washed with 3-4 mL of a 0.1 M trisodium citrate buffer, pH 3.0. The column is then washed generously with PBS. Then, 1 mL of rabbit antiserum is diluted with 1 mL PBS, and the resulting solution is slowly applied to the column. The column is washed with 15 mL PBS and eluted with a 0.1 M trisodium citrate buffer, pH 3.0. Three fractions of 2.2 mL are collected in 15 mL graduated tubes containing 0.8 mL of 1 M Tris-HCl buffer, pH 8.5. The purified rabbit IgG antibodies are stored at 4° C. in the presence of 0.01% sodium azide.

Antibody Specificity

To ensure accuracy in the ELISA measurement of CYP 2C9 phenotyping, the antibodies must have specificity for their individual molecules, with little or no recognition of other derivatives. To ensure their selectivity an ELISA is performed with standard solutions of (s)-ibuprofen metabolites and other structurally similar compounds.

Results

Positive creation of antibodies against (s)-ibuprofen and 2-carboxyibuprofen can be seen by antibody titers of 30,000-100,000 as determined by the ELISA, strong precipitation lines after double immunodiffusion in agar plates of antisera and derivatives conjugated to rabbit serum albumin, and low cross-reactivity with other mephenytoin derivatives. These results constitute positive conditions for the development of a competitive antigen ELISA according to the methods described in the above section entitled Materials and Methods.

### EXAMPLE I

### A Competitive Antigen ELISA for CYP 2C9 Phenotyping

Buffers and water without additives are filtered through 0.45 μM millipore filters and kept for one week, except the substrate buffer which is freshly prepared. BSA, antibodies, Tween™ 20 and horse radish peroxidase are added to buffers and water just prior to use.

Preparation of Urine Samples

Urine samples are usually collected four hours after ingestion of (s)-ibuprofen and stored at −20° C. as 1-mL aliquots in 1.5 mL microtubes. For the ELISA, the urine samples are diluted with isotonic sodium phosphate buffer, pH 7.5 (310 mosM) to give concentrations of (s)-ibuprofen or 2-carboxyibuprofen no higher than 3×10⁻⁶ M in the microtiter plate wells. Just prior to the ELISA, samples are mixed in a 1:1 ratio (e.g. 100 μl:100 μl) with either the (s)-ibuprofen-HRP or the 2-carboxyibuprofen-HRP conjugate (12 mg ml⁻¹).

Standard Solutions of 2-carboxyibuprofen or (s)-ibuprofen for ELISA

A 100 mL stock solution of (s)-ibuprofen or 2-carboxyibuprofen at concentrations of 6.00×10⁻⁴ M is prepared in the 310 mosM sodium phosphate buffer, pH 7.5 (IPB) in a 100 mL volumetric flask. The solution is stirred to insure complete solubilization.

The stock solutions are stored as 1 mL aliquots at −20° C. On the day of the ELISA, one aliquot is thawed and warmed up at room temperature. The following standard solutions (Table 2) of the above compounds are prepared:

TABLE 2
Standard Solutions

| Standard # | [Compound] | | Composition |
|---|---|---|---|
| 1 | 6.00 × 10⁻⁴ | M | Stock Solution |
| 2 | 2.00 × 10⁻⁴ | M | 200 μL S1 + 400 μ0L IPB |
| 3 | 1.12 × 10⁻⁴ | M | 200 μL S1 + 868 μL IPB |
| 4 | 6.00 × 10⁻⁵ | M | 100 μL S1 + 900 μL IPB |
| 5 | 3.56 × 10⁻⁵ | M | 60 μL S1 + 951 μL IPB |
| 6 | 2.00 × 10⁻⁵ | M | 100 μL S2 + 900 μL IPB |
| 7 | 1.12 × 10⁻⁵ | M | 100 μL S3 + 900 μL IPB |
| 8 | 6.00 × 10⁻⁶ | M | 100 μL S4 + 900 μL IPB |
| 9 | 3.56 × 10⁻⁶ | M | 100 μL S5 + 900 μL IPB |
| 10 | 2.00 × 10⁻⁶ | M | 100 μL S6 + 900 μL IPB |
| 11 | 1.12 × 10⁻⁶ | M | 100 μL S7 + 900 μL IPB |
| 12 | 6.00 × 10⁻⁷ | M | 100 μL S8 + 900 μL IPB |
| 13 | 3.56 × 10⁻⁷ | M | 100 μL S9 + 900 μL IPB |
| 14 | 2.00 × 10⁻⁷ | M | 100 μL S10 + 900 μL IPB |
| 15 | 1.12 × 10⁻⁷ | M | 100 μL S11 + 900 μL IPB |
| 16 | 6.00 × 10⁻⁸ | M | 100 μL S12 + 900 μL IPB |
| 17 | 3.56 × 10⁻⁸ | M | 100 μL S13 + 900 μL IPB |
| 18 | 2.00 × 10⁻⁸ | M | 100 μL S14 + 900 μL IPB |
| 19 | 2.00 × 10⁻⁹ | M | 100 μL S15 + 900 μL IPB |
| 20 | 2.00 × 10⁻¹⁰ | M | 100 μL S15 + 900 μL IPB |
| 21 | 2.00 × 10⁻¹¹ | M | 100 μL S15 + 900 μL IPB |
| 22 | 2.00 × 10⁻¹² | M | 100 μL S15 + 900 μL IPB |
| 23 | 2.00 × 10⁻¹³ | M | 100 μL S15 + 900 μL IPB |

ELISA Conditions

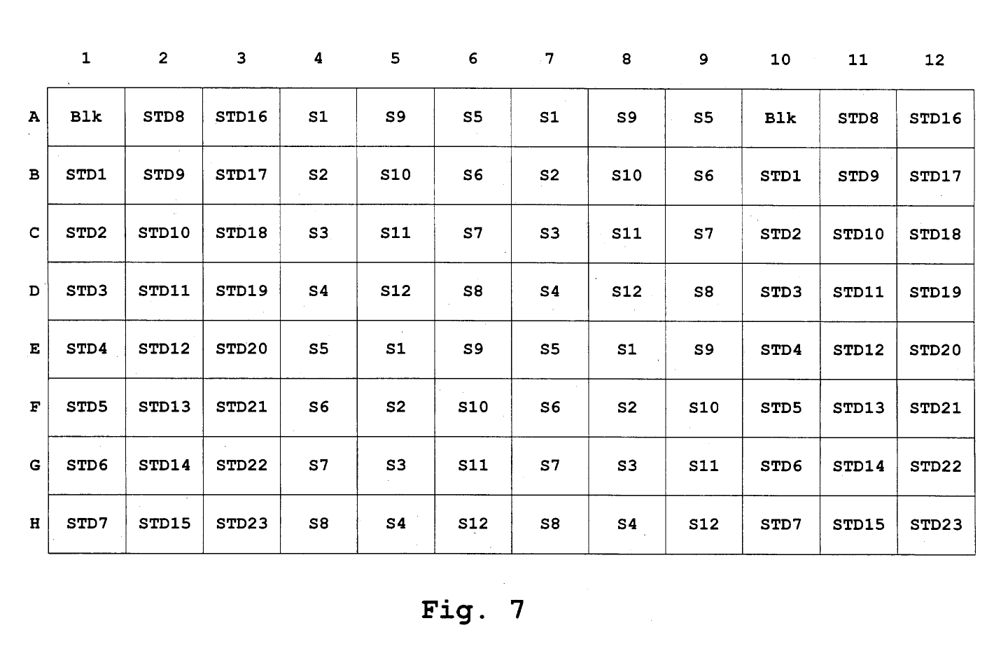

Wells of the ELISA plate are washed with a Nunc-Immuno wash 12 washer. Then, 16 mL of a solution of 6.6 μg ml⁻¹ of isolated IgG antibodies is prepared in a 100 mM sodium carbonate buffer, pH 9.6, and 150 μL of this solution is pipetted in each well of a microtiter plate using a eight channel pipet (Brinkmann Transferpette™-8 50-200 μL) and 200 μL Flex tips from Brinkmann). After coating the wells with antibodies at 4° C. for 20 h, the wells are washed 3 times with the isotonic sodium phosphate buffer containing 0.05% Tween™ 20 (IPBT) and properly drained by inverting the plate and absorbing the liquid on piece of paper towel. Next, 30 mL of a solution of a IPBT solution containing 1% BSA is prepared and 150 μL of this solution is pipetted in each well using a eight channel pipet (Brinkmann Transferpette™-8 50-200 μL) and 200 μL yellow tips (Sarstedt yellow tips for P200 Gilson Pipetman). After 3 h at room temperature, the wells were washed 3 times with IPBT solution and drained. Then, 400 μl of sample or standard for determination of 2-carboxyibuprofen or (s)-ibuprofen are prepared (as described in previous sections) in 1.5 mL microtubes using Sarstedt yellow tips and a P200 Gilson Pipetman. Each sample/standard (200 μL) is pipetted in duplicate in a Falcon 96 well microtest tissue culture plate according to the pattern shown in FIG. 7, using Sarstedt yellow tips and a P200 Gilson Pipetman. Using an eight channel pipet (Brinkmann Transferpette™-8 50-200 μL) and changing the tips of the eight channel pipet (200 μL Flex tips from Brinkmann) at each row, 150 μL of sample/standard are transferred in the corresponding wells of a 96 well ELISA microtiter plate coated with antibodies. After the addition of the samples and standards, the microtiter plates are covered and left standing at room temperature for 2 h. While the plate is left standing the substrate buffer without the hydrogen peroxide and o-phenylenediamine hydrochloride is prepared (25 mM citric acid and 50 mM sodium phosphate dibasic buffer, pH 5.0). The microtiter plate is washed 3 times with the IPBT solution and 3 times with a 0.05% Tween™ 20 solution and drained. Next, 50 μL of hydrogen peroxide and 40 mg of o-phenylenediamine are added to the substrate buffer. One hundred and fifty microliters (150 μL) of the substrate buffer solution is then added to each well using an eight channel pipet (Brinkmann Transferpette™-8 50-200 μL) and 200 μL Flex tips (Brinkmann). The microtiter plate is covered and shaken for 25-30 min at room temperature and the enzymatic reaction is stopped by adding 50 μL/well of a 2.5 M HCl solution using an eight channel pipet (Brinkmann Transferpette™-8 50-200 μL) and 200 μL Flex tips Brinkmann). After gently shaking for 3 min, the absorbance is read at 490 nm with a microplate reader.

### EXAMPLE II

### Determination of (s)-ibuprofen and 2-carboxyibuprofen in Urine Samples with the ELISA Kit

The contents of an ELISA kit for determining CYP 2C9 phenotype are exemplified in Table 3.

TABLE 3
Content of the ELISA kit and Conditions of Storage

| | | | | Storage |
|---|---|---|---|---|
| Item | Unit | State | Amt. | Conditions |
| Tween ™ 20 | 1 vial | liquid | 250 μL/vial | 4° C. |
| H₂O₂ | 1 vial | liquid | 250 μL/vial | 4° C. |
| (s)-ibuprofen-HRP | 1 vial | liquid | 250 μL/vial | 4° C. |
| 2-carboxyibuprofen | 1 vial | liquid | 250 μL/vial | 4° C. |
| HRP | | | | |
| Buffer* A | 4 vials | Solid | 0.8894 g/vial | 4° C. |
| Buffer* B | 6 vials | Solid | 1.234 g/vial | 4° C. |
| Buffer* C | 6 vials | Solid | 1.1170 g/vial | 4° C. |
| Buffer* D | 6 vials | Solid | 0.8082 g/vial | 4° C. |
| Plate ((s)-ibuprofen- | 2 | Solid | — | 4° C. |
| Ab) | | | | |
| Plate (2-carboxy- | 2 | Solid | — | 4° C. |
| ibuprofen-Ab) | | | | |
| Buffer* E | 6 vials | Solid | 0.9567 g/vial | −20° C. |
| Standards | 14 vials | Liquid | 200 μL | −20° C. |
| ((s)-ibuprofen) | | | | |
| Standards (2- | 14 vials | Liquid | 200 μL | −20° C. |
| carboxyibuprofen) | | | | |
| 1 N NaOH | 1 bottle | Liquid | 15 mL | 20° C. |
| 1 N HCl | 1 bottle | Liquid | 15 mL | 20° C. |

*Composition of all buffers is described in Table 10

Solutions

Buffer A: Dissolve the content of 1 vial A/25 mL water.

Buffer B: Dissolve the content of 1 vial B/100 mL water.

Buffer C: Dissolve the content of one vial C/50 mL water. Add 25 mL of Tween™ 20.

Buffer D: Dissolve the content of one vial D/25 mL water. Add 25 mL of Tween™ 20.

0.05% Tween™ 20: Add 25 mL of Tween™ 20 to a 100 mL erlenmeyer flask containing 50 mL of water.

2.5N HCl: 41.75 mL of 12 N HCl/200 mL water. Store in a 250 mL glass bottle.

(s)-ibuprofen-HRP conjugate: Add 9 mL of Buffer C to a 15 mL glass test tube. Add 90 μL of (s)-ibuprofen-HRP stock solution.

2-carboxyibuprofen-HRP conjugate: Add 9 mL of Buffer C to a 15 mL glass test tube. Add 90 μL of 2-carboxyibuprofen-HRP stock solution.

Buffer E—H₂O₂: Dissolve the contents of 1 vial E-substrate/50 mL water. Add 25 μL of a 30% H₂O₂ solution (prepared fresh).

Dilutions of Urine Samples for the Determinations of [(s)-ibuprofen-HRP] and [2-carboxyibuprofen-HRP] by ELISA

The dilutions of urine samples required for determinations of (s)-ibuprofen and 2-carboxyibuprofen are a function of the sensitivity of the competitive antigen ELISA and of (s)-ibuprofen and 2-carboxyibuprofen concentrations in urine samples. It is suggested to dilute the urine samples by a factor so (s)-ibuprofen and 2-carboxyibuprofen are about 3×10⁻⁶ M in the well of the microtiter plate (see table 4).

TABLE 4
Microtube #

| Dilution Factor | 20x | 40x | 50x | 80x | 100x | 150x | 200x | 400x |
|---|---|---|---|---|---|---|---|---|
| Solution | 1 | 2 | 3 | 4 | 5 | 6 | 7 | 8 |
| Urine Sample (μL)^{a} | 500 | 250 | 200 | 125 | 100 | 66.7 | 50 | 25 |
| 10× diluted | | | | | | | | |
| Buffer B (μL) | 500 | 750 | 800 | 875 | 900 | 933.3 | 950 | 975 |

^{a}Vortex the microtubes containing the urine sample before pipetting.

Store the diluted urine samples at −20° C. in a box for microtubes.

Determination of [(s)-ibuprofen] and [2-carboxyibuprofen] in Diluted Urine Samples by ELISA

Precautions

The HRP substrate (p-nitrophenolphosphate) is carcinogenic. Wear surgical gloves when handling Buffer E (substrate buffer). Each sample is determined in duplicate. An excellent pipetting technique is required. When this technique is mastered the absorbency values of duplicates should be within less than 5%. Buffers C, D, E are freshly prepared. Buffer E—H₂O₂ is prepared just prior to pipetting in the microtiter plate wells.

Preparation of Samples

Table 5 is prepared with a computer and printed. This table shows the contents of each well of a 96 well microtiter plate. The name of the urine sample (or number) is entered at the corresponding well positions in Table 5. The dilution factor (D.F.) of each urine sample is selected and entered at the corresponding position in Table 5. The dilution of each urine sample with buffer B is entered at the corresponding position in Table 5: for example, for a D.F. of 100 (100 μL of 10×diluted urine sample+900 μL buffer B), 100/900 is entered. See “Dilutions of Urine Samples . . . ” procedure described above for the preparation of the different dilutions. The different dilutions of the urine samples are prepared in 1.5 mL microtubes using a styrofoam support for 100 microtubes. Standard solutions of concentrations indicated in Table 6 are preferably provided with the kit of the present invention. Table 7 is prepared with a computer and printed. Using a styrofoam support (100 microtubes), the following 48 microtubes are prepared in the order as indicated in Table 7.

TABLE 5
Positions of Blanks, Control and Urine Samples in a Microtiter Plate

| Sample | Well # | D.F. | Dil. |
|---|---|---|---|
| Blank | 1-2 | — | |
| Control | 3-4 | — | |
| S1 | 5-6 | — | |
| S2 | 7-8 | — | |
| S3 | 9-10 | — | |
| S4 | 11-12 | — | |
| S5 | 13-14 | — | |
| S6 | 15-16 | — | |
| S7 | 17-18 | — | |
| S8 | 19-20 | — | |
| S9 | 21-22 | — | |
| S10 | 23-24 | — | |
| S11 | 25-26 | — | |
| S12 | 27-28 | — | |
| S13 | 29-30 | — | |
| S14 | 31-32 | — | |
| S15 | 33-34 | — | |
| 1 | 35-36 | | |
| 2 | 37-38 | | |
| 3 | 39-40 | | |
| 4 | 41-42 | | |
| 5 | 43-44 | | |
| 6 | 45-46 | | |
| 7 | 47-48 | | |
| Control | 49-50 | | — |
| 8 | 51-52 | | |
| 9 | 53-54 | | |
| 10 | 55-56 | | |
| 11 | 57-58 | | |
| 12 | 59-60 | | |
| 13 | 61-62 | | |
| 14 | 63-64 | | |
| 15 | 65-66 | | |
| 16 | 67-68 | | |
| 17 | 69-70 | | |
| Control | 71-72 | | — |
| 18 | 73-74 | | |
| 19 | 75-76 | | |
| 20 | 77-78 | | |
| 21 | 79-80 | | |
| 22 | 81-82 | | |
| 23 | 83-84 | | |
| 24 | 85-86 | | |
| 25 | 87-88 | | |
| 26 | 89-90 | | |
| 27 | 91-92 | | |
| 28 | 93-94 | | |
| Blank | 95-96 | | — |

TABLE 6
Standard Solutions of (s)-ibuprofen and 2-carboxyibuprofen
(Diluted with Buffer B)

| Standard | (s)-ibuprofen | Standard | 2-carboxyibuprofen |
|---|---|---|---|
| 1 | 1.12 × 10⁻⁴ M | 1 | 1.12 × 10⁻⁴ M |
| 2 | 6.00 × 10⁻⁵ M | 2 | 6.00 × 10⁻⁵ M |
| 3 | 3.56 × 10⁻⁵ M | 3 | 3.56 × 10⁻⁵ M |
| 4 | 2.00 × 10⁻⁵ M | 4 | 2.00 × 10⁻⁵ M |
| 5 | 6.00 × 10⁻⁶ M | 5 | 6.00 × 10⁻⁶ M |
| 6 | 3.56 × 10⁻⁶ M | 6 | 3.56 × 10⁻⁶ M |
| 7 | 2.00 × 10⁻⁶ M | 7 | 2.00 × 10⁻⁶ M |
| 8 | 1.12 × 10⁻⁶ M | 8 | 1.12 × 10⁻⁶ M |
| 9 | 6.00 × 10⁻⁷ M | 9 | 6.00 × 10⁻⁷ M |
| 10 | 3.56 × 10⁻⁷ M | 10 | 3.56 × 10⁻⁷ M |
| 11 | 2.00 × 10⁻⁷ M | 11 | 2.00 × 10⁻⁷ M |
| 12 | 1.12 × 10⁻⁷ M | 12 | 1.12 × 10⁻⁷ M |
| 13 | 6.00 × 10⁻⁸ M | 13 | 6.00 × 10⁻⁸ M |
| 14 | 3.56 × 10⁻⁸ M | 14 | 3.56 × 10⁻⁸ M |
| 15 | 2.00 × 10⁻⁸ M | 15 | 2.00 × 10⁻⁸ M |

TABLE 7
Content of Microtubes for CYP 2C9 phenotyping ELISA

| Tube # | Sample | Content |
|---|---|---|
| 1 | Blank | Buffer B |
| 2 | Control | Buffer B |
| 3 | S1 | (s)-ibuprofen/2-carboxyibuprofen |
| 4 | S2 | (s)-ibuprofen/2-carboxyibuprofen |
| 5 | S3 | (s)-ibuprofen/2-carboxyibuprofen |
| 6 | S4 | (s)-ibuprofen/2-carboxyibuprofen |
| 7 | S5 | (s)-ibuprofen/2-carboxyibuprofen |
| 8 | S6 | (s)-ibuprofen/2-carboxyibuprofen |
| 9 | S7 | (s)-ibuprofen/2-carboxyibuprofen |
| 10 | S8 | (s)-ibuprofen/2-carboxyibuprofen |
| 11 | S9 | ((s)-ibuprofen/2-carboxyibuprofen |
| 12 | S10 | (s)-ibuprofen/2-carboxyibuprofen |
| 13 | S11 | (s)-ibuprofen/2-carboxyibuprofen |
| 14 | S12 | (s)-ibuprofen/2-carboxyibuprofen |
| 15 | S13 | (s)-ibuprofen/2-carboxyibuprofen |
| 16 | S14 | (s)-ibuprofen/2-carboxyibuprofen |
| 17 | S15 | (s)-ibuprofen/2-carboxyibuprofen |
| 18 | 1 | Dil. Urine |
| 19 | 2 | Dil. Urine |
| 20 | 3 | Dil. Urine |
| 21 | 4 | Dil. Urine |
| 22 | 5 | Dil. Urine |
| 23 | 6 | Dil. Urine |
| 24 | Control | Buffer B |
| 25 | 7 | Dil. Urine |
| 26 | 8 | Dil. Urine |
| 27 | 9 | Dil. Urine |
| 28 | 10 | Dil. Urine |
| 29 | 11 | Dil. Urine |
| 30 | 12 | Dil. Urine |
| 31 | 13 | Dil. Urine |
| 32 | 14 | Dil. Urine |
| 33 | 15 | Dil. Urine |
| 34 | 16 | Dil. Urine |
| 35 | 17 | Dil. Urine |
| 36 | Control | Buffer B |
| 37 | 18 | Dil. Urine |
| 38 | 19 | Dil. Urine |
| 39 | 20 | Dil. Urine |
| 40 | 21 | Dil. Urine |
| 41 | 22 | Dil. Urine |
| 42 | 23 | Dil. Urine |
| 43 | 24 | Dil. Urine |
| 44 | 25 | Dil. Urine |
| 45 | 26 | Dil. Urine |
| 46 | 27 | Dil. Urine |
| 47 | 28 | Dil. Urine |
| 48 | Blank | Buffer B |

Conditions of the ELISA

Starting from the last row, 50 μL/well of (s)-ibuprofen-HRP ((s)-ibuprofen/2-carboxyibuprofen) conjugate are added. Next are added 50 μL/well of diluted urine samples in duplicate, standards, and blanks with a micropipet (0-200 μL), starting from well #96 (see Table 8). The plate is covered and mixed gently by vortexing for several seconds. The plate is left at room temperature for 3 h. Then, the wells are washed 3 times with 100 μL/well Buffer C, using a microtiter plate washer. The wells are then washed 3 times with 100 μL/well 0.05% Tween™-20 solution. Next, 150 μL/well of Buffer E—H₂O₂ (prepared just prior to pipetting in the microtiter plate wells) are added. The plate is shaken for 20-30 min at room temperature using an orbital shaker. After shaking, 50 μL/well of a 2.5N HCl solution are added. The plate is shaken again 3 min with the orbital shaker at room temperature. The absorbance of the wells are read with a microtiter plate reader at 490 nm. The sheet of data is printed and properly labelled.

Calculation of the [(s)-ibuprofen] and [2-carboxyibuprofen] in Urine Samples from the Data

Table 8 is drawn with a computer. Using the data sheet of the microtiter plate reader, the average absorbance values of blanks, controls (no free hapten present), standards and samples are entered in Table 8. The calibration curve is drawn on a semi-logarithmic plot (absorbance at 490 nm as a function of the standard concentrations) using sigma-plot (or other plot software). The [(s)-ibuprofen] (or [2-carboxyibuprofen]) is found in the microtiter well of the unknowns from the calibration curve and entered in the data in Table 9. The [(s)-ibuprofen] (or [2-carboxyibuprofen]) of the unknown is multiplied by the dilution factor and the result is entered in the corresponding cell of Table 9.

TABLE 8
Average Absorbance Values of Samples
in the Microtiter Plate

| Sample | Well # | A₄₉₀ |
|---|---|---|
| Blank | 1-2 | |
| Control | 3-4 | |
| S1 | 5-6 | |
| S2 | 7-8 | |
| S3 | 9-10 | |
| S4 | 11-12 | |
| S5 | 13-14 | |
| S6 | 15-16 | |
| S7 | 17-18 | |
| S8 | 19-20 | |
| S9 | 21-22 | |
| S10 | 23-24 | |
| S11 | 25-26 | |
| S12 | 27-28 | |
| S13 | 29-30 | |
| S14 | 31-32 | |
| S15 | 33-34 | |
| 1 | 35-36 | |
| 2 | 37-38 | |
| 3 | 39-40 | |
| 4 | 41-42 | |
| 5 | 43-44 | |
| 6 | 45-46 | |
| 7 | 47-48 | |
| Control | 49-50 | |
| 8 | 51-52 | |
| 9 | 53-54 | |
| 10 | 55-56 | |
| 11 | 57-58 | |
| 12 | 59-60 | |
| 13 | 61-62 | |
| 14 | 63-64 | |
| 15 | 65-66 | |
| 16 | 67-68 | |
| 17 | 69-70 | |
| Control | 71-72 | |
| 18 | 73-74 | |
| 19 | 75-76 | |
| 20 | 77-78 | |
| 21 | 79-80 | |
| 22 | 81-82 | |
| 23 | 83-84 | |
| 24 | 85-86 | |
| 25 | 87-88 | |
| 26 | 89-90 | |
| 27 | 91-92 | |
| 28 | 93-94 | |
| Blank | 95-96 | |

TABLE 9
(s)-ibuprofen and 2-carboxyibuprofen Concentrations
in Urine Samples

| | | | |
|---|---|---|---|
| Sample | D.F. | [(s)-ibuprofen] | [(s)-ibuprofen] × D.F. |

```
  1
  2
  3
  4
  5
  6
  7
  8
  9
10
11
12
13
14
15
16
17
18
19
20
21
22
23
24
25
26
27
28
29
```

TABLE 10
Composition of the Different Buffers

| | | | Conc. | |
|---|---|---|---|---|
| Buffer | pH | Composition | (mM) | [P] (mM) |
| A | 7.50 | 0.15629 g/100 mL NaH₂PO₄ | 11.325 | 71.424 |
| | | 1.622 g/100 mL Na₂HPO₄.7H₂O | 60.099 | |
| | | 1.778 g/100 mL (total weight) | | |
| B | 7.50 | 0.1210191 g/100 mL NaH₂PO₄ | 8.769 | 49.999 |
| | | 1.11309 g/100 mL Na₂HPO₄.7H₂O | 41.23 | |
| | | 1.2341 g/100 mL (total weight) | | |
| C | 7.50 | 1 g/100 mL BSA | 8.769 | 49.999 |
| | | 0.1210191 g/100 mL NaH₂PO₄ | 41.23 | |
| | | 1.11309 g/100 mL Na₂HPO₄.7H₂O | | |
| | | 2.2341 g/100 mL (total weight) | | |
| D | 7.50 | 2 g/100 mL BSA | 8.769 | 49.999 |
| | | 0.1210191 g/100 mL NaH₂PO₄ | 41.23 | |
| | | 1.11309 g/100 mL Na₂HPO₄.7H₂O | | |
| | | 3.2341 g/100 mL (total weight) | | |
| E | 5.00 | 0.52508 g/100 mL of citric acid | 25 | — |
| | | 1.34848 g/100 mL Na₂HPO₄.7H₂O | 50 | |
| | | 40 mg/100 mL of o-phenylenedi- | | |
| | | amine hydrochloride | | |
| | | 1.913567 g/100 mL (total weight) | | |

Discussion

In the form of a kit, the present invention provides a convenient and effective tool for use in both a clinical and laboratory environment. The kit of the present invention is particularly suited for use by a physician or other qualified personnel in a clinic, whereby a fast and accurate result can be easily obtained. According to an embodiment of the present invention, a ready-to-use kit is provided for fast and accurate determination of an individual's CYP 2C9 phenotype. Preferably, a kit of the present invention includes a microtest plate having a plurality of wells for receiving biological samples to be tested for metabolite concentrations indicative of a CYP 2C9-specific phenotypic determinant. The microtest plate may be pre-bound with antibodies specific to the metabolites of interest. The kit may further include suitable substrates and buffers, such as those exemplified in Table 3.

As a result of the convenience and ease of use of is ELISA and/or kit of the present invention, a physician is provided with a tool for use in the individualization of treatment. A quick and accurate determination of an individual's CYP 2C9 phenotype will allow a physician to consider this information before prescribing a treatment regime. In this manner, a method of individualizing treatment is also provided. In essence, a CYP 2C9 phenotype characterization, according to the present invention, can serve as a drug response profile specific to drugs known to be metabolized by CYP 2C9 for the individual phenotyped. Furthermore, the ELISA and/or kit of the present invention may be used to screen individuals for their susceptibility to carcinogens or for their phenotypic compatibility with a particular drug known to metabolized completely or in part by CYP 2C9.

The present invention provides a convenient and effective tool for use in both a clinical and laboratory environment. The present invention is particularly suited for use by a physician in a clinic, whereby phenotypic determinants of CYP 2C9 can be quickly and easily obtained. According to an embodiment of the present invention, a ready-to-use kit is provided for fast and accurate determination of at least CYP 2C9 determinants. The assay system and kit preferably employ antibodies specific to a plurality of substrates and/or forms thereof on a suitable substrate allowing for detection of the preferred substrates in a biological sample of an individual after consumption of a corresponding substrate (or probe substrate). In accordance with a preferred embodiment of the present invention, the kit of the present invention will provide means to determine metabolic determinants for at least CYP 2C9. The assay system and method of the present invention may be provided in a plurality of forms including but not limited to an ELISA assay, a high-throughput ELISA assay or a dipstick based ELISA assay.

The ELISA and/or kit of an embodiment of the present invention includes antibodies specific to preferred metabolites, substrates and/or forms thereof known to be acted on by the CYP 2C9 metabolic pathway immobilized on a suitable substrate to detect the presence of the preferred metabolites, substrates and/or forms thereof in a biological sample of an individual after consumption of a corresponding probe substrate.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims. All references cited within this application are hereby incorporated by reference.

What is claimed is:

1. A method of characterizing a CYP 2C9-specific phenotype, said method comprising:
a) administering to an individual a probe substrate known to be metabolized by a CYP 2C9 metabolic pathway;
b) detecting concentrations of said probe substrate and/or forms thereof in a biological sample obtained from said individual at a predetermined time after said administering of said probe substrate; and
c) characterizing a phenotypic determinant based on said concentrations of said probe substrate and/or forms thereof which is indicative of said CYP 2C9 phenotype.

2. The method of claim 1 wherein said probe substrate is (s)-ibuprofen.

3. The method of claim 2 wherein said probe substrate and/or forms of said probe substrate include (s)-ibuprofen and 2-carboxyibuprofen.

4. The method of claim 3 wherein said phenotypic determinant is characterized according to a molar ratio of concentrations of said probe substrate and/or forms of said probe substrate, as calculated by:
$$\frac{[ ( s ) - ibuprofen ]}{[ 2 - carboxyibuprofen ]} .$$

5. The method of claim 1, wherein said step of detecting concentrations of said probe substrate and/or forms thereof includes a ligand-binding assay, whereby said assay includes treating said biological sample with binding molecules specific to each of said probe substrates and/or forms thereof.

6. The method of claim 5, wherein said step of detecting concentrations of said probe substrate and/or forms thereof further includes measuring the absorbency of a binding molecule-ligand conjugate for each of said probe substrate and/or forms thereof.

7. The method of claim 5, wherein said binding molecules specific to each of said probe substrates and/or forms thereof are antibodies.

8. The method of claim 1, wherein said biological sample is a urine sample.

9. The method of claim 1, wherein said phenotypic determinant is indicative of said individual's susceptibility to a carcinogen induced disease.

10. The method of claim 9, wherein said carcinogen-induced disease is cancer.

11. The method of claim 1, wherein said phenotypic determinant is indicative of said individual's responsiveness to a drug known to be metabolized by CYP 2C9.

12. The method of claim 1 for use in selecting a drug treatment regime for said individual.

13. The method of claim 1 for use in screening individuals for a CYP 2C9 phenotype requirement prior to participation in a clinical trial.

14. The method of claim 1 for use in individualization of treatment wherein said treatment is influenced by CYP 2C9 metabolism.

15. A competitive enzyme linked immunosorbent assay (ELISA) method for determining a CYP 2C9 phenotype, which comprises using at least two antibodies specific to (s)-ibuprofen and 2-carboxyibuprofen, respectively, to determine the amount of each of (s)-ibuprofen and 2-carboxyibuprofen in a biological sample obtained from an individual treated with (s)-ibuprofen; wherein a molar ratio based on amounts of the (s)-ibuprofen to 2-carboxyibuprofen is indicative of a CYP 2C9 phenotype of said individual.

16. The ELISA method of claim 15, wherein said biological sample is a urine sample.

17. The ELISA method of claim 15, wherein said determined CYP 2C9 phenotype of said individual provides an indication of said individual's susceptibility to a carcinogen-induced disease.

18. The method of claim 15, wherein said disease is cancer.

19. The ELISA method of claim 15, wherein said CYP 2C9 phenotype provides a drug response profile for said individual.

20. The method of claim 18 for use in selecting a drug treatment regime for said individual.

21. A competitive enzyme linked immunosorbent assay (ELISA) kit for determining a CYP 2C9 phenotype of an individual, which comprises at least two antibodies each specific to a probe substrate and/or at least one other form thereof for detecting the molar ratio of said probe substrate and/or at least one other form thereof in a biological sample of an individual after consuming a dose of said probe substrate wherein said molar ratio is indicative of said CYP 2C9 phenotype.

22. The competitive enzyme linked immunosorbent assay (ELISA) kit of claim 21 for determining a CYP 2C9 phenotype of an individual, said kit comprising at least one plate having a plurality of microwells for receiving biological samples obtained from said individual; said microwells having an antibody coating selected from said at least two antibodies.

23. The competitive enzyme linked immunosorbent assay (ELISA) kit of claim 21 wherein said at least two antibodies are specific to (s)-ibuprofen and 2-carboxyibuprofen, respectively.

24. The competitive enzyme linked immunosorbent assay (ELISA) kit of claim 21, further comprising:
a) a known amount of (s)-ibuprofen-horseradish peroxidase conjugate wherein a standard calibration curve is obtained; and
b) a known amount of 2-carboxyibuprofen-horseradish peroxidase conjugate wherein a standard calibration curve is obtained.

25. The kit of claim 21, for use in determining the susceptibility of said individual to a carcinogen-induced disease.

26. The kit of claim 21, for use in determining a drug response profile specific to said individual.

27. The kit of claim 21, for use in selecting a drug treatment regime for said individual.

28. The method of claim 5, wherein said binding molecules are monoclonal antibodies.

29. The method of claim 5, wherein said binding molecules are polyclonal antibodies.

30. The competitive antigen enzyme linked immunosorbent assay (ELISA) method of claim 15 wherein said at least two antibodies are monoclonal antibodies.

31. The competitive antigen enzyme linked immunosorbent assay (ELISA) method of claim 15 wherein said at least two antibodies are polyclonal antibodies.

32. The competitive ELISA kit of claim 21 wherein said at least two antibodies are monoclonal antibodies.

33. The competitive ELISA kit of claim 21 wherein said at least two antibodies are polyclonal antibodies.

34. A (s)-ibuprofen derivative as illustrated in FIG. 3.

35. A 2-carboxyibuprofen derivative as illustrated in FIG. 4.

36. The (s)-ibuprofen derivative of claim 34 for use in raising antibodies having an affinity for (s)-ibuprofen.

37. The 2-carboxyibuprofen derivative of claim 35 for use in raising antibodies having an affinity for 2-carboxyibuprofen.

38. The (s)-ibuprofen derivative of claim 34 for use in detecting (s)-ibuprofen in a biological sample.

39. The 2-carboxyibuprofen derivative of claim 35 for use in detecting 2-carboxyibuprofen in a biological sample.

40. The antibodies of claim 36 for use in an ELISA for determining a CYP 2C9 phenotype.

41. The antibodies of claim 37 for use in an ELISA for determining a CYP 2C9 phenotype.

42. The method of claim 1 wherein said probe substrate is losartan.

43. The method of claim 2 wherein said probe substrate and/or forms of said probe substrate include losartan and E-3174.

44. The method of claim 3 wherein said phenotypic determinant is characterized according to a molar ratio of concentrations of said probe substrate and/or forms of said probe substrate, as calculated by:
$$\frac{[ losartan ]}{[ E - 3174 ]} .$$

45. A competitive enzyme linked immunosorbent assay (ELISA) method for determining a CYP 2C9 phenotype, which comprises using at least two antibodies specific to losartan and E-3174, respectively, to determine the amount of each of losartan and E-3174 in a biological sample obtained from an individual treated with losartan; wherein a molar ratio based on amounts of the losartan to losartan is indicative of a CYP 2C9 phenotype of said individual.

46. The ELISA method of claim 45, wherein said biological sample is a urine sample.

47. The ELISA method of claim 45, wherein said determined CYP 2C9 phenotype of said individual provides an indication of said individual's susceptibility to a carcinogen-induced disease.

48. The method of claim 45, wherein said disease is cancer.

49. The ELISA method of claim 45, wherein said CYP 2C9 phenotype provides a drug response profile for said individual.

50. The method of claim 48 for use in selecting a drug treatment regime for said individual.

51. The competitive enzyme linked immunosorbent assay (ELISA) kit of claim 21 wherein said at least two antibodies are specific to losartan and E-3174, respectively.

52. The competitive enzyme linked immunosorbent assay (ELISA) kit of claim 21, further comprising:
a) a known amount of losartan-horseradish peroxidase conjugate wherein a standard calibration curve is obtained; and
b) a known amount of E-3174-horseradish peroxidase conjugate wherein a standard calibration curve is obtained.

53. The competitive antigen enzyme linked immunosorbent assay (ELISA) method of claim 45 wherein said at least two antibodies are monoclonal antibodies.

54. The competitive antigen enzyme linked immunosorbent assay (ELISA) method of claim 45 wherein said at least two antibodies are polyclonal antibodies.

55. A losartan derivative as illustrated in FIG. 5.

56. A E-3174 derivative as illustrated in FIG. 6.

57. The losartan derivative of claim 55 for use in raising antibodies having an affinity for losartan.

58. The E-3174 derivative of claim 56 for use in raising antibodies having an affinity for E-3174.

59. The losartan derivative of claim 55 for use in detecting losartan in a biological sample.

60. The E-3174 derivative of claim 56 for use in detecting E-3174 in a biological sample.

61. The antibodies of claim 57 for use in an ELISA for determining a CYP 2C9 phenotype.

62. The antibodies of claim 58 for use in an ELISA for determining a CYP 2C9 phenotype.
